(19) United States
(12) Patent Application Publication
(10) Pub. No.: US 2026/0029409 A1
(43) Pub. Date: Jan. 29, 2026

(54) MARKERS FOR DIAGNOSING INFECTIONS

(71) Applicant: MeMed Diagnostics Ltd., Tirat HaCarmel (IL)

(72) Inventors: Michal ROSENBERG, Haifa (IL); Alon ANGEL, Haifa (IL); Oded SHAHAM, Haifa (IL); Roy NAVON, Tel-Aviv (IL); Einav SIMON, Atlit (IL); Eran EDEN, Haifa (IL); Eran REINER, Kiryat Ata (IL)

(73) Assignee: MeMed Diagnostics Ltd., Tirat HaCarmel (IL)

(21) Appl. No.: 18/997,109

(22) PCT Filed: Jul. 20, 2023
(86) PCT No.: PCT/IL2023/050761

§ 371 Date: Jan. 20, 2025

Related U.S. Application Data
(60) Provisional application No. 63/390,701, filed on Jul. 20, 2022.

(51) Int. Cl. G01N 33/68

(52) U.S. Cl. CPC G01N 33/6893; G01N 2333/54; G01N 2800/26

(57) ABSTRACT

A method of diagnosing an infectious disease, including determining the severity of the disease, in a subject comprising measuring an expression level of at least one protein selected from the group consisting of TSG-14, AGER, ANG-2 and ST2 in a sample of the subject; and diagnosing the disease based on said expression level. Kits for carrying out the diagnosis are also disclosed.

### RELATED APPLICATION/S

This application claims the benefit of priority of U.S. Provisional Patent Application No. 63/390,701 filed on 20 Jul. 2022, the contents of which are incorporated herein by reference in their entirety.

### FIELD AND BACKGROUND OF THE INVENTION

The present invention, in some embodiments thereof, relates to the identification of signatures and determinants associated with bacterial and viral infections.

Disease assessment is one of the most important tasks in management of infectious disease patients. Complement to determining infection etiology, predicting patient prognosis may affect various aspects of patient management including treatment, diagnostic tests (e.g., microbiology, blood chemistry, radiology etc.), and admission. Timely identification of patients with higher chance for poor prognosis may result in more aggressive patient management procedures including for example, intensive care unit (ICU) admission, advanced therapeutics, invasive diagnostics or surgical intervention, which could reduce complications and mortality.

Additional background art includes WO 2013/117746, WO 2016/024278, WO2018/060998 and WO2018/060999.

### SUMMARY OF THE INVENTION

According to an aspect of the present invention, there is provided a method of diagnosing an infectious disease in a subject comprising:
(a) measuring an expression level of at least one protein selected from the group consisting of Tumor necrosis factor-inducible gene 14 protein (TSG-14), Advanced glycosylation end product-specific receptor (AGER), Angiogpoietin-2 (ANG-2) and Interleukin 1 receptor-like 1 (ST2) in a sample of the subject; and
(b) diagnosing the disease based on the expression level.

According to embodiments of the invention,
(i) when the expression level of TSG-14 is at least 2 fold higher than a level in a control sample, a severe infectious disease is ruled in;
(ii) when the expression level of AGER is at least 2 fold higher than a level in a control sample, a severe infectious disease is ruled in;
(iii) when the expression level of ANG-2 is at least 2 fold higher than a level in a control sample, a severe infectious disease is ruled in; and/or
(iv) when the expression level of ST2 is is at least 3 fold higher than a level in a control sample, a severe infectious disease is ruled in.

According to embodiments of the invention, the diagnosing comprises determining the severity of the infectious disease.

According to embodiments of the invention, the expression level of TSG-14 is below about 930 μg/ml, a severe infectious disease is ruled out;
(ii) when the expression level of AGER is below about 960 μg/ml, a severe infectious disease is ruled out;
(iii) when the expression level of ANG-2 is below about 1800 μg/ml, a severe infectious disease is ruled out; and/or
(iv) when the expression level of ST2 is below about 28,000 μg/ml, a severe infectious disease is ruled out.

According to embodiments of the invention, (i) when the expression level of TSG-14 is above about 6000 μg/ml, a severe infectious disease is ruled in;
(ii) when the expression level of AGER is above about 3200 μg/ml, a severe infectious disease is ruled in;
(iii) when the expression level of ANG-2 is above about 5000 μg/ml, a severe infectious disease is ruled in; and/or
(iv) when the expression level of ST2 is above about 140,000 μg/ml, a severe infectious disease is ruled in.

According to embodiments of the invention, the at least one protein comprises at least two proteins.

According to embodiments of the invention, the at least two proteins comprise ANG-2 and AGER; AGER and ST2; or ANG-2 and ST2.

According to embodiments of the invention, the method further comprises measuring an expression of at least one additional protein selected from the group consisting of IL-6, IL-10 and MR-proADM and diagnosing the infection based on the expression level of the at least one additional protein in combination with the expression level of the at least one protein.

According to embodiments of the invention,
(i) when the expression level of IL-10 is below 0.17 pg/ml, a severe infectious disease is ruled out;
(ii) when the expression level of IL-6 is below 9.8 pg/ml, a severe infectious disease is ruled out; and/or
(iii) when the expression level of MR-proADM is below 0.6 nmol/L, a severe infectious disease is ruled out.

According to embodiments of the invention,
(i) when the expression level of IL-10 is above 68 μg/ml, a severe infectious disease is ruled in;
(ii) when the expression level of IL-6 is above 56 μg/ml, a severe infectious disease is ruled in; and/or
(iii) when the expression level of MR-proADM is above 1.9 nmol/L, a severe infectious disease is ruled in.

According to embodiments of the invention:
(i) when the expression level of IL-10 is at least 3 fold higher than a level in a control sample, a severe infectious disease is ruled in;
(ii) when the expression level of IL-6 is is at least 2 fold higher than a level in a control sample, a severe infectious disease is ruled in.

According to embodiments of the invention, the method further comprises measuring an expression level of IP-10 and diagnosing the infection based on the expression level of IP-10 in combination with the expression level of the at least one protein.

According to embodiments of the invention, the method further comprises measuring an expression level of IP-10 and diagnosing the infection based on the expression level of IP-10 in combination with the expression level of the at least two proteins.

According to an aspect of the present invention, there is provided a method of diagnosing an infectious disease of a subject, comprising measuring the amount of soluble urokinase plasminogen activator receptor (suPAR) and the amount of at least one determinant selected from the group consisting of Interferon gamma-induced protein 10 (IP-10) and Interleukin-6 (IL-6) in a sample of the subject, wherein a combined amount of the suPAR and the determinant is indicative of the severity of the infection.

According to embodiments of the invention, when the amount of suPAR is above a predetermined level and the amount of IP-10 is above a predetermined level, the infection is classified as severe.

According to embodiments of the invention, when the amount of suPAR is below a predetermined level and the amount of IP-10 is below a predetermined level, the infection is classified as non-severe.

According to embodiments of the invention, when the amount of suPAR is above a predetermined level and the amount of IL-6 is above a predetermined level, the infection is classified as severe.

According to embodiments of the invention, when the amount of suPAR is below a predetermined level and the amount of IL-6 is below a predetermined level, the infection is classified as non-severe.

According to embodiments of the invention, the method further comprises measuring an expression level of TRAIL and/or CRP.

According to embodiments of the invention, the method further comprises measuring all the components of a clinical index selected from the group consisting of NEWS, NEWS 2, MEWS APACHE I, APACHE II, APACHE III, CURB-65, SMART-COP, SAPS II, SAPS III, PIM2, CMM, SOFA, qSOFA, MPM, RIFLE, CP, MODS, LODS, Rochester criteria, Philadelphia Criteria, Milwaukee criteria and Ranson score.

According to embodiments of the invention, the method further comprises measuring the level of at least one additional protein set forth in Tables 5, 6 or 7.

According to embodiments of the invention, the infection is a viral infection.

According to embodiments of the invention, the infection is a bacterial infection.

According to embodiments of the invention, the subject shows symptoms of an infectious disease.

According to embodiments of the invention, the subject does not show symptoms of an infectious disease.

According to embodiments of the invention, the subject does not have a chronic non-infectious disease.

According to embodiments of the invention, the sample is whole blood or a fraction thereof.

According to embodiments of the invention, the fraction comprises cells selected from the group consisting of lymphocytes, monocytes and granulocytes.

According to embodiments of the invention, the fraction comprises serum or plasma.

According to embodiments of the invention, the level of no more than 10 proteins is used to diagnose the infection.

According to embodiments of the invention, no more than 6 proteins are measured to diagnose the infection.

According to embodiments of the invention, the diagnosing an infection comprises determining a severity of the infection.

According to an aspect of the present invention, there is provided a kit for diagnosing an infection comprising detection reagents which specifically detect a first determinant selected from the group consisting of IP-10, MR-proADM, IL-6 and IL-10 and a second determinant selected from the group consisting of TSG-14, AGER, ANG-2 and ST2.

According to an aspect of the present invention, there is provided a kit for diagnosing an infection comprising detection reagents which specifically at least two determinants selected from the group consisting of TSG-14, AGER, ANG-2 and ST2.

According to embodiments of the invention, the determinant is IP-10.

According to an aspect of the present invention, there is provided a kit for determining the severity of an infection comprising:
(i) an antibody which binds specifically to a determinant selected from the group consisting of IP-10 and IL-6; and
(ii) an antibody which binds specifically to suPAR, wherein the kit comprises no more than ten antibodies.

According to embodiments of the invention, the kit further comprises a detection reagent which specifically detects IP-10.

According to embodiments of the invention, the kit further comprises detection reagents which specifically detect TRAIL.

According to embodiments of the invention, the kit further comprises detection reagents which specifically detect CRP.

According to embodiments of the invention, the detection reagents are antibodies.

According to embodiments of the invention, the at least one of the antibodies is attached to a detectable moiety.

According to embodiments of the invention, the at least one of the antibodies is a monoclonal antibody.

According to embodiments of the invention, the at least one of the antibodies is attached to a solid support.

According to embodiments of the invention, the kit comprises detection reagents that specifically detect no more than 10 protein markers.

According to embodiments of the invention, the kit comprises detection reagents that specifically detect no more than 6 protein markers.

According to an aspect of the present invention, there is provided a method of treating a subject having an infectious disease comprising:
(a) diagnosing the infection according to any one of claims 1-29; and
(b) treating the subject according to the diagnosis of the infection.

According to embodiments of the invention, when a severe infection is ruled in, at least one of the following treatments is used: hospitalization; placement in intensive care; mechanical ventilation; non-invasive ventilation, ECMO, renal replacement therapy, cardiac catheterization, Antibiotic treatment, vasopressor therapy and/or treatment of last resort.

According to embodiments of the invention, the subject shows symptoms of an infectious disease.

According to embodiments of the invention, the symptoms comprise fever.

According to an aspect of the present invention there is provided a method of distinguishing between a viral and bacterial infection in a subject comprising:
(a) measuring an expression level of a combination of proteins in a blood sample of the subject, the combination being presented in Groups 1 or 2; and
(b) determining whether the infection is bacterial or viral based on the expression level.

According to an aspect of the present invention there is provided a method of determining the severity of an infectious disease in a subject comprising:
(a) measuring an expression level of a combination of proteins in a blood sample of the subject, the combination being in Group 3 or Group 4; and
(b) determining the severity based on the expression level.

According to an aspect of the present invention there is provided a method of diagnosing an infectious disease in a subject comprising:
(a) measuring an expression level of a combination of proteins in a blood sample of the subject, the combination being in Groups 5 or 6; and
(b) determining whether the disease is infectious or non-infectious based on the expression level.

According to embodiments of the invention, the method further comprises measuring the level of at least one additional protein set forth in Tables 5 or 7.

According to embodiments of the invention, the method further comprises measuring the level of at least one additional protein set forth in Tables 6 or 7.

According to embodiments of the invention, the method further comprises determining the severity of the infection.

According to embodiments of the invention, the determining the severity of the infection is effected by measuring the level of at least one protein set forth in Table 6.

According to embodiments of the invention, the method further comprises measuring the level of at least one additional protein set forth in Table 5.

According to embodiments of the invention, the subject shows symptoms of an infectious disease.

According to embodiments of the invention, the subject does not show symptoms of an infectious disease.

According to embodiments of the invention, the subject does not have a chronic non-infectious disease.

According to embodiments of the invention, the sample is whole blood or a fraction thereof.

According to embodiments of the invention, the fraction comprises cells selected from the group consisting of lymphocytes, monocytes and granulocytes.

According to embodiments of the invention, the fraction comprises serum or plasma.

According to embodiments of the invention, the level of no more than 10 proteins is used to classify the infection.

According to embodiments of the invention, no more than 5 proteins are measured to determine the infection type.

According to an aspect of the present invention there is provided a kit for diagnosing an infection type comprising detection reagents which specifically detect each of the proteins of the combinations set forth in Groups 1-6.

According to embodiments of the invention, the detection reagents are antibodies.

According to embodiments of the invention, at least one of the antibodies is attached to a detectable moiety.

According to embodiments of the invention, at least one of the antibodies is a monoclonal antibody.

According to embodiments of the invention, at least one of the antibodies is attached to a solid support.

According to embodiments of the invention, the kit comprises detection reagents that specifically detect no more than 10 protein markers.

According to embodiments of the invention, the kit comprises detection reagents that specifically detect no more than 6 protein markers.

According to an aspect of the present invention there is provided a method of treating a subject having an infectious disease comprising:
(a) classifying the infection type according to any one of claims 1-16; and
(b) treating the subject according to the classification of the infection, wherein when a severe viral infection is ruled in, at least one of the following treatments is used: treatment with an antiviral agent; hospitalization; placement in intensive care; mechanical ventilation; and/or treatment of last resort.

According to embodiments of the invention, the antiviral agent is selected from the group consisting of Molnupiravir, Paxlovid and Remdesivir.

According to embodiments of the invention, the subject shows symptoms of an infectious disease.

According to embodiments of the invention, the symptoms comprise fever.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF SPECIFIC EMBODIMENTS OF THE INVENTION

The present invention, in some embodiments thereof, relates to the identification of signatures and determinants associated with bacterial and viral infections.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Differentiating between bacterial and viral infections is a daily clinical challenge. Recent publications have shown that the host response to severe infection display an inflammatory ‘bacterial’ pattern, regardless of the underlying infectious etiology, even at the case of an underlying viral infection (Tang, B. M., Nature Communications 10, 3422. doi:10.1038/s41467-019-11249-y; Dunning, J., et al Nature Immunology 19(6):625-635. doi:10.1038/s41590-018-0111-5).

By carrying out large clinical studies, the present inventors have now discovered unique proteins present in the blood which serve as markers of infection severity. The present inventors propose diagnosing subjects and making appropriate treatment decisions based on the expression level of such markers.

Whilst further reducing the invention to practice, the present inventors uncovered combinations of such markers which are able to classify infections in terms of severity with a very high degree of accuracy. Such proteins can be combined with additional protein determinants which are able to distinguish between bacterial and viral infections. This enables a highly detailed diagnosis of infections in a relatively short amount of time.

Thus, according to an aspect of the present invention there is provided a method of diagnosing an infectious disease in a subject comprising:
(a) measuring an expression level of at least one protein selected from the group consisting of Tumor necrosis factor-inducible gene 14 protein (TSG-14), Advanced glycosylation end product-specific receptor (AGER), Angiogpoietin-2 (ANG-2) and Interleukin 1 receptor-like 1 (ST2) in a sample of the subject; and
(b) diagnosing the disease based on the expression level.

The term “diagnosing” as used herein refers to determining presence or absence of an infection, classifying an infection or a symptom thereof, determining a severity of the infection, monitoring infection progression, forecasting an outcome of an infection and/or determining prospects of recovery.

According to an embodiment of this aspect of the present invention, the diagnosing comprises determining or classifying a severity of the infection.

Information regarding particularly relevant protein markers which may be used for diagnosing (and more specifically for determining severity) is provide in Table 1A, herein below.

TABLE 1A

| Gene Symbol | UniProt ID | Biomarker |
|---|---|---|
| ANGPT2 | O15123 | Angiopoietin-2; ANG-2 |
| IL1RL1/DER4/ST2/T1 | Q01638 | Interleukin-1 receptor-like 1; Protein ST2; |
| AGER/RAGE | Q15109 | Advanced glycosylation end product-specific receptor; |
| | | Receptor for advanced glycosylation end products |
| IL6/IFNB2 | P05231 | Interleukin-6; IL-6; B-cell stimulatory factor 2 (BSF-2); |
| | | CTL differentiation factor (CDF); Hybridoma growth |
| | | factor |
| | | Interferon beta-2 (IFN-beta-2) |
| PTX3/TNFAIP5/TSG14 | P26022 | Pentraxin-related protein PTX3; Tumor necrosis factor |
| | | alpha-induced protein 5 (TNF alpha-induced protein 5); |
| | | Tumor necrosis factor-inducible gene 14 protein (TSG-14) |
| IL-10 | P22301 | Interleukin-10; IL-10; Cytokine synthesis inhibitory |
| | | factor (CSIF) |
| ADM/AM | P35318 | MR-proADM; Pro-adrenomedullin; Adrenomedullin |
| | | (AM); Proadrenomedullin N-20 terminal peptide |
| | | (PAMP; ProAM-N20) |

For example, the protein markers disclosed in Table 1A may be used to rule in a severe infection or rule in a non-severe infection. Each of the markers in Table 1A are increased in severe infection as compared to non-severe infection as further detailed herein below.

Additionally, or alternatively, the protein markers disclosed in Table 1A may be used to rule out a severe infection or rule out a non-severe infection.

In some embodiments, at least one of the protein markers disclosed in Table 1A may be used to rule in a severe viral infection or rule out a severe viral infection.

Additionally, or alternatively, at least one of the protein markers disclosed in Table 1A may be used to rule in a non-severe viral infection or rule out a non-severe virl infection.

In some embodiments, at least one of the protein markers disclosed in Table 1A may be used to rule in a severe bacterial infection or rule out a severe bacterial infection.

Additionally, or alternatively, at least one of the proteins disclosed in Table 1A may be used to rule in a non-severe bacterial infection or rule out a non-severe bacterial infection. When the level of any of the above disclosed proteins are above a predetermined amount, a severe infection may be ruled in.

The predetermined level is the amount (i.e. level) of (or a function of the amount of) the protein in a control sample derived from one or more subjects who do not have an infection (i.e., healthy, and or non-infectious individuals) or who do not have a severe infection (i.e., subjects who have a non-severe infection). In a further embodiment, such subjects are monitored and/or periodically retested for a diagnostically relevant period of time (“longitudinal studies”) following such test to verify continued absence of infection. Such period of time may be one day, two days, two to five days, five days, five to ten days, ten days, or ten or more days from the initial testing date for determination of the reference value. Furthermore, retrospective measurement of protein levels in properly banked historical subject samples may be used in establishing these reference values, thus shortening the study time required.

A reference value can also comprise the amounts of proteins derived from subjects who show an improvement as a result of treatments and/or therapies for the infection. A reference value can also comprise the amounts of proteins derived from subjects who have confirmed infection by known techniques.

According to a specific embodiment, when the expression level of TSG-14 is below about 930 μg/ml, a severe infectious disease is ruled out. Other exemplary thresholds for TSG-14 that may be used below which a severe infection is ruled out include below about 910 μg/ml, below about 890 μg/ml or below about 870 μg/ml.

Other exemplary thresholds for TSG-14 that may be used below which a severe infection is ruled out include below about 500 μg/ml, below about 370 μg/ml, below about 270 μg/ml or below about 170 μg/ml.

According to another embodiment, when the expression level of AGER is below about 960 μg/ml, a severe infectious disease is ruled out. Other exemplary thresholds for AGER that may be used below which a severe infection is ruled out include below about 930 μg/ml, below about 900 μg/ml or below about 880 μg/ml.

Other exemplary thresholds for AGER that may be used below which a severe infection is ruled out include below about 710 μg/ml, below about 610 μg/ml, below about 590 μg/ml or below about 560 μg/ml.

According to another embodiment, when the expression level of ANG-2 is below about 1800 μg/ml, a severe infectious disease is ruled out. Other exemplary thresholds for ANG-2 that may be used below which a severe infection is ruled out include below about 1500 μg/ml, below about 1300 μg/ml or below about 1100 μg/ml.

Other exemplary thresholds for ANG-2 that may be used below which a severe infection is ruled out include below about 1200 μg/ml, below about 1000 μg/ml, below about 990 μg/ml or below about 920 μg/ml.

According to another embodiment, when the expression level of ST2 is below about 28,000 μg/ml, a severe infectious disease is ruled out. Other exemplary thresholds for ST2 that may be used below which a severe infection is ruled out include below about 25,000 μg/ml, below about 20,000 μg/ml or below about 15,000 μg/ml.

Other exemplary thresholds for ST2 that may be used below which a severe infection is ruled out include below about 23,000 μg/ml, below about 18,000 μg/ml, below about 15,000 μg/ml or below about 13,000 μg/ml.

According to another embodiment, when the expression level of IL-10 is below about 0.17 μg/ml, a severe infectious disease is ruled out. Other exemplary thresholds for IL-10 that may be used below which a severe infection is ruled out include below about 0.16 μg/ml, below about 0.15 μg/ml or below about 0.14 μg/ml.

According to another embodiment, when the expression level of IL-6 is below about 9.8 μg/ml, a severe infectious disease is ruled out. Other exemplary thresholds for IL-6 that may be used below which a severe infection is ruled out include below about 9.6 μg/ml, below about 9.4 μg/ml or below about 9.2 μg/ml.

Other exemplary thresholds for IL-6 that may be used below which a severe infection is ruled out include below about 5.9 μg/ml, below about 4.6 μg/ml, below about 4.3 μg/ml or below about 3.5 μg/ml.

According to another embodiment, when the expression level of TSG-14 is above about 6000 μg/ml, a severe infectious disease is ruled in. Other exemplary thresholds for TSG-14 that may be used above which a severe infection is ruled in include above about 7000 μg/ml, above about 8000 μg/ml or above about 10,000 μg/ml.

Other exemplary thresholds for TSG-14 that may be used above which a severe infection is ruled in include above about 7,100 μg/ml, above about 9,300 μg/ml, above about 15,000 μg/ml, above about 21,000 μg/ml, above about 30,000 μg/ml or above about 39,000 μg/ml.

According to still another embodiment, when the expression level of TSG-14 is increased by at least two fold or even 2.5 fold over the baseline of TSG-14 (e.g. when the subject has a non-severe infection, or when the subject is healthy or when the subject is non-infectious), a severe infection may be ruled in.

According to another embodiment, when the expression level of AGER is above about 3200 μg/ml, a severe infectious disease is ruled in. Other exemplary thresholds for AGER that may be used above which a severe infection is ruled in include above about 3300 μg/ml, above about 3500 μg/ml or above about 4,000 μg/ml.

Other exemplary thresholds for AGER that may be used above which a severe infection is ruled in include above about 3,900 μg/ml, above about 5,500 μg/ml, above about 6,700 μg/ml, above about 10,000 μg/ml, above about 13,000 μg/ml, above about 14,000 μg/ml.

According to still another embodiment, when the expression level of AGER is increased by at least two fold over the baseline of AGER (when the subject (e.g. when the subject has a non-severe infection, or when the subject is healthy or when the subject is non-infectious), a severe infection may be ruled in.

According to another embodiment, when the expression level of ANG-2 is above about 5000 μg/ml, a severe infectious disease is ruled in. Other exemplary thresholds for ANG-2 that may be used above which a severe infection is ruled in include above about 6,000 μg/ml, above about 7,000 μg/ml or above about 8,000 μg/ml.

Other exemplary thresholds for ANG-2 that may be used above which a severe infection is ruled in include above about 5,800 μg/ml, above about 7,000 μg/ml, above about 10,000 μg/ml, above about 14,000 μg/ml, above about 17,000 μg/ml, above about 20,000 μg/ml.

According to still another embodiment, when the expression level of ANG-2 is increased by at least two fold over the baseline of ANG-2 (e.g. when the subject has a non-severe infection, or when the subject is healthy or when the subject is non-infectious), a severe infection may be ruled in.

According to another embodiment, when the expression level of ST2 is above about 140,000 μg/ml, a severe infectious disease is ruled in. Other exemplary thresholds for ST2 that may be used above which a severe infection is ruled in include above about 150,000 μg/ml, above about 170,000 μg/ml or above about 200,000 μg/ml.

Other exemplary thresholds for ST2 that may be used above which a severe infection is ruled in include above about 180,000 μg/ml, above about 230,000 μg/ml, above about 390,000 μg/ml, above about 500,000 μg/ml, above about 770,000 μg/ml.

According to still another embodiment, when the expression level of ST2 is increased by at least three fold over the baseline of ST2 (e.g. when the subject has a non-severe infection, or when the subject is healthy or when the subject is non-infectious), a severe infection may be ruled in.

According to another embodiment, when the expression level of IL-10 is above about 68 μg/ml, a severe infectious disease is ruled in. Other exemplary thresholds for IL-10 that may be used above which a severe infection is ruled in include above about 70 μg/ml, above about 72 μg/ml or above about 75 μg/ml.

Other exemplary thresholds for IL-10 that may be used above which a severe infection is ruled in include above about 88 μg/ml, above about 130 μg/ml, above about 210 μg/ml, above about 270 μg/ml, above about 350 μg/ml, above about 1,900 μg/ml.

According to still another embodiment, when the expression level of IL-10 is increased by at least three fold over the baseline of IL-10 (e.g. when the subject has a non-severe infection, or when the subject is healthy or when the subject is non-infectious), a severe infection may be ruled in.

According to another embodiment, when the expression level of IL-6 is above about 56 μg/ml, a severe infectious disease is ruled in. Other exemplary thresholds for IL-6 that may be used above which a severe infection is ruled in include above about 57 μg/ml, above about 60 μg/ml or above about 65 μg/ml.

Other exemplary thresholds for IL-6 that may be used above which a severe infection is ruled in include above about 75 μg/ml, above about 130 μg/ml, above about 260 μg/ml, above about 410 μg/ml, above about 500 μg/ml, above about 1,000 μg/ml.

According to still another embodiment, when the expression level of IL-6 is increased by at least two fold over the baseline of IL-6 (e.g. when the subject has a non-severe infection, or when the subject is healthy or when the subject is non-infectious), a severe infection may be ruled in.

The term “classifying the severity” refers to assignment of the severity of the disease which may in one embodiment, relate to the probability to experience certain adverse events (e.g. death, hospitalization or admission to ICU) to an individual. Thus, the classification may also be used to prognose the outcome of a patient with an infectious disease. Classifying the severity of the disease may be effected on a binary level (severe/non-severe) or may be effected on non-binary level (e.g. based on numerical values, such as severity categories 1, 2, 3 etc.).

In one embodiment, the severity can be classified according to the WHO ordinal scale of disease stratification, NEWS (National Early Warning Score), SOFA (Sequential Organ Failure Assessment) score and qSOFA (Quick SOFA) Score for Sepsis.

In one embodiment, the term “severe” refers to an infection that will have at least one of the following outcomes: will require vasopressor therapy, will require intubation with mechanical ventilation, will require non-invasive ventilation, will be admitted to the intensive care unit and/or predicted to die within 14 days.

The term “non-severe”, in one embodiment, refers to an infection that will not require vasopressor therapy, will not require intubation with mechanical ventilation, will not require non-invasive ventilation, will not be admitted to the intensive care unit and/or will not be predicted to die within 14 days.

Particular combinations of the above disclosed markers that have shown a very high degree of accuracy in determining severity of infection include ANG-2 and AGER; AGER and ST2; and ANG-2 and ST2.

The present inventors have shown that the combinations may be highly relevant for determining severity in particular subgroups of patients. For example, the combination of AGER and ANG-2 may be used for determining severity viral diseases (e.g., for ruling out or ruling in a severe viral disease). An exemplary threshold of AGER is 1758 ng/ml and for ANG-2 999 ng/ml. The combination of ST2 and ANG-2 may be used for determining severity of bacterial diseases (e.g. ruling out a severe bacterial disease). An exemplary threshold of ST2 is 37,554 ng/ml and for ANG-2 is 2,545 ng/ml. The combination of ST2 and AGER may be used for determining severity of bacterial diseases (e.g., ruling in a severe bacterial disease). An exemplary threshold of AGER is 202,000 ng/ml and for ANG-2 is 5,650 ng/ml.

The determinants listed in Table 1A may be combined with IP-10 to bring about a more accurate diagnosis of the infection. For example, the following pairs of markers are contemplated. TSG-14 and IP-10; AGER and IP-10; ANG-2 and IP-10 and ST2 and IP-10. The following triplets are contemplated for diagnosing infections: IP-10, ANG-2 and AGER; IP-10, AGER and ST2; and IP-10, ANG-2 and ST2.

Additional combinations of proteins that may be used to diagnose infections and more specifically to determine the severity of infection are provided herein below: MR-proADM and IL-6; MR-proADM and IL-10; MR-proADM and TSG-14; MR-proADM and AGER; MR-proADM and ANG-2; and MR-proADM and ST2.

IL-10 and IL-6; IL-10 and TSG-14; IL-10 and AGER; IL-10 and ANG-2; and IL-10 and ST2.

MR-proADM and IL-6; IL-6 and TSG-14; IL-6 and AGER; IL-6 and ANG-2; and MR-IL-6 and ST2.

The present inventors have found that the expression level of the markers TRAIL, CRP and IP-10 are particularly relevant for distinguishing between bacterial and viral infections. Accordingly, combinations of this triplet with at least one of the markers listed in Table 1A are also contemplated. For example, the combinations TRAIL, CRP, IP-10 and TSG-14; TRAIL, CRP, IP-10 AGER, TRAIL, CRP, IP-10 and ANG-2; and TRAIL, CRP, IP-10 and ST2.

The level of TRAIL increases in viral infections (as compared to non-infectious diseases), and decreases in bacterial infections (as compared to non-infectious diseases).

Thus, when the level of TRAIL is above a predetermined level, it is indicative that the infection is a viral infection and a viral infection may be ruled in (or a bacterial infection may be ruled out).

When the level of TRAIL is below a predetermined level, it is indicative that the infection is a bacterial infection and a bacterial infection may be ruled in (or a viral infection may be ruled out).

For example, a bacterial infection may be ruled out if the polypeptide concentration of TRAIL determined is higher than a pre-determined first threshold value. Optionally, the method further includes determining if a subject has a viral infection (i.e., ruling in a viral infection). A viral infection is ruled in if the polypeptide concentration of TRAIL is higher than a pre-determined second threshold value.

In another specific embodiment the invention includes determining if a subject does not have a viral infection (i.e. ruling out a viral infection). A viral infection is ruled out if the polypeptide concentration of TRAIL determined is lower than a pre-determined first threshold value. Optionally, the method further includes determining if a subject has a bacterial infection (i.e., ruling in a bacterial infection). A bacterial infection is ruled in if the polypeptide concentration of TRAIL is lower than a pre-determined second threshold value.

More specifically, TRAIL levels of 100-1000 μg/ml are usually indicative of a viral infection, while 0-85 μg/ml are usually indicative of a bacterial infection. Bacterial infection can usually be ruled in if TRAIL levels are lower than 85 μg/ml, 70 μg/ml, 60 μg/ml or more preferably 50, 40, 30 or 20 μg/ml, and ruled out if TRAIL levels are higher than 100, 120, 140 or preferably 150 μg/ml.

The level of CRP typically increases in infections (as compared to non-infectious diseases), with the level of CRP being higher in bacterial infections as opposed to viral infections.

Thus, when the level of CRP is above a predetermined level, it is indicative that the infection is a bacterial infection and a bacterial infection may be ruled in (or a viral infection may be ruled out).

The level of IP-10 increases in infections (as compared to non-infectious diseases), with the level of IP-10 being higher in viral infections as opposed to bacterial infections.

Thus, when the level of IP-10 is above a predetermined level, it is indicative that the infection is a viral infection and a viral infection may be ruled in (or a bacterial infection may be ruled out).

When the level of IP-10 is below a predetermined level, it is indicative that the infection is a bacterial infection and a bacterial infection may be ruled in (or a viral infection may be ruled out).

IP-10 levels of 300-2000 μg/ml are usually indicative of a viral infection, while 160-860 μg/ml are usually indicative of a bacterial infection.

Additional proteins that may be measured together with at least one, at least two, or at least three of the markers listed in Table 1A for measuring severity of infections include any of those listed in Table 6. Combinations of markers that may be included for measuring severity are listed as belonging to Groups 3 or 4.

Additional proteins that may be measured for distinguishing between bacterial and viral infections include any of those listed in Table 5. Combinations of markers that may be included for distinguishing between bacterial and viral are listed as belonging to Groups 1 or 2.

Additional proteins that may be measured for distinguishing between infectious and non-infectious diseases include any of those listed in Table 7. Combinations of markers that may be included for distinguishing between infectious and non-infectious are listed as belonging to Groups 5 or 6.

Additional factors that can be incorporated for diagnosing infection and in particular for classifying severity include epidemiological information, symptom assessment, and traditional laboratory results as summarized in Table to, herein below.

TABLE 1B

| Epidemiological | Main complaint & | Timing & Clinical | Physical examination and |
|---|---|---|---|
| information | clinical syndrome | scores | ancillary tests |
| Age | Main complaint: | Time from | Vital signs: |
| Gender | Confusion | symptom onset | Temperature |
| Re-admission | Cough | Time from | Heart rate |
| status | Urinary | arrival to: | Respiratory rate |
| Arrival status | retention | Antibiotics | Mean arterial |
| (ambulance Vs. | Changes in | administration | pressure |
| self-admitted) | urination | Fluid | Blood pressure |
| Comorbidities: | Abdominal | administration | Oxygen saturation |
| Diabetes | pain | Source | Central venous |
| mellitus (type | Altered mental | control | pressure |
| 1/2) | status | Admission | Central venous |
| Hypertension | Shortness of | ICU | oxygen saturation |
| Heart failure | breath | transfer | Capillary refill |
| Coronary | Chest pain | Clinical scores: | time |
| artery disease | Fever/hypothermia | Glasgow Coma | Lab: |
| Cardiomyopathies | Weakness | Scale (GCS) | White blood count |
| Peripheral | Vomiting | Systemic | Hemoglobin |
| vascular | Diarrhea | inflammatory | Absolute |
| disease | Rash | response | neutrophil count |
| Chronic | Skin lesions | syndrome | (ANC) |
| obstructive | Articular | (SIRS) | Bands % |
| pulmonary | tenderness/ | sequential | Monocyte |
| disease | pain/swelling | organ failure | distribution width |
| Peptic ulcer | Clinical syndrome: | assessment | Platelets |
| disease | Lower | (SOFA) | D-Dimer |
| Asthma | Respiratory | qSOFA | PT |
| Interstitial lung | Tract Infection | National Early | PTT |
| disease | Pneumonia | Warning | Bilirubin total |
| Pulmonary | Upper | Score (NEWS) | Creatinine |
| embolism | Respiratory | Acute | Creactive protein |
| Pulmonary | Tract Infection | Physiology and | Erythrocyte |
| hypertension | Urinary Tract | Chronic Health | sedimentation rate |
| Bronchiectasis | Infection | Evaluation II | Blood urea |
| Cystic fibrosis | Pyelonephritis | (APACHII2) | nitrogen |
| Cerebrovascular | Bacteremia | Modified Early | procalcitonin |
| accident | Soft tissue | Warning Score | Transaminases |
| Myocardial | infection | (MEWS) | Aminotransferases |
| infarction | Necrotizing | | Albumin |
| Immunosuppression | fasciitis | | international |
| Chronic kidney | Encephalitis | | normalized ratio |
| disease | Meningitis | | Glucose |
| Chronic liver | Septicemia | | Sodium, chloride |
| disease | Osteomyelitis | | Hemoglobin |
| Obesity | Abscess | | troponin |
| (BMI >30 | Enterocolitis | | B-type natriuretic |
| kg/m²) | Infective | | peptide |
| Overweight | Endocarditis | | pH |
| (BMI 25 to 29 | Septic arthritis | | Lactate |
| kg/m2) | Fever of | | PaO2/FiO2 |
| Sleep apnea | unknown | | PaCo2 |
| Osteoarthritis | origin | | Bicarbonate |
| Malignancy | Prosthetic | | Microbiology |
| Depression | device | | confirmation: |
| Schizophrenia | infections | | Positive antigen |
| spectrum | | | test |
| disorders | | | Nasal swab PCR |
| Dementia | | | Blood/urine/sputum/soft |
| Connective | | | tissue |
| tissue disease | | | cultures |
| Pregnancy or | | | Imaging findings: |
| recent | | | Atelectasis |
| pregnancy | | | ARDS |
| Smoking | | | Consolidations |
| (current and | | | Pleural effusion |
| former) | | | Intrabdominal |
| Sickle cell | | | abscess |
| disease or | | | |
| thalassemia | | | |
| Solid organ or | | | |
| blood stem cell | | | |
| transplantation | | | |
| Substance use | | | |
| disorders | | | |
| Tuberculosis | | | |
| Human | | | |
| immunodeficiency | | | |
| virus | | | |
| Use of | | | |
| corticosteroids | | | |
| or other | | | |
| immunosuppressive | | | |
| medications | | | |
| Number of | | | |
| comorbidities | | | |

Both suPAR (see for example WO 2019/162334) and IP-10 (see for example WO 2021/152595) are markers known to correlate with disease severity.

The present inventors have now shown (see Example 3) that by calculating a score based on the combination of these two markers, the level of accuracy for predicting a severe outcome of an infectious disease is significantly increased. The present inventors propose that the combined measurement should assist physicians in assessing a patient's risk profile, enabling better informed management decisions.

Thus, according to another aspect of the present invention there is provided a method of diagnosing an infectious disease of a subject, comprising measuring the amount of soluble urokinase plasminogen activator receptor (suPAR) and the amount of at least one determinant selected from the group consisting of Interferon gamma-induced protein 10 (IP-10) and Interleukin-6 (IL-6) in a sample of the subject, wherein a combined amount of the suPAR and the determinant is indicative of the severity of the infection.

The protein suPAR (UniProt ID: Q03405, NCBI Accession no. AAK31795 and isoforms of the receptor, NP_002650, 003405, NP_002650, NP_001005376) is the soluble portion of Urokinase-type Plasminogen Activator Receptor (uPAR), which is released by cleavage of the GPI anchor of membrane-bound uPAR. suPAR is a family of glycosylated proteins consisting of full length suPAR (277 amino acids (1-277)) and suPAR fragments D1 (1-83), and D2D3 (84-277) generated by urokinase cleavage or human airway trypsin-like protease, D1 (1-87) and D2D3 (88-277) generated by MMP cleavage, D1 (1-89) and D2D3 (90-277) also generated by urokinase cleavage or human airway trypsin-like protease, D1 (1-91) and D2D3 (92-277) generated by cleavage by plasmin. In one embodiment, the severity determination is carried out by generating a score based on the amount of both suPAR and IP-10 (i.e. the combination of suPAR and IP-10).

The combination refers to any mathematical combination of suPAR and IP-10.

In one embodiment, the score is an increasing function of the amount of suPAR and an increasing function of the amount of IP-10. In this case, when the score is above a predetermined level a severe infection is ruled in, the predetermined level being based on the amount of both suPAR and IP-10 in non-severely infected subjects.

The score may be a monotonically increasing function of the amount of suPAR and a monotonically increasing function of the amount of IP-10. In one embodiment, the function is linear.

In another embodiment, the score may be a decreasing function of the amount of suPAR and a decreasing function of the amount of IP-10. In this case, when the score is below a predetermined level, a severe infection is ruled in, the predetermined level being based on the amount of both suPAR and IP-10 in non-severely infected subjects.

The score may be a monotonically decreasing function of suPAR and a monotonically decreasing function of the amount of IP-10. In one embodiment, the function is linear.

In one embodiment, the score is based on the ratio of suPAR:IP-10.

In still another embodiment, the score is based on the ratio of IP-10:suPAR.

In one embodiment, the severity determination is carried out by generating a score based on the amount of both suPAR and IL-6 (i.e. the combination of suPAR and IL-6). The combination refers to any mathematical combination of suPAR and IL-6.

In one embodiment, the score is an increasing function of the amount of suPAR and an increasing function of the amount of IL-6. In this case, when the score is above a predetermined level a severe infection is ruled in, the predetermined level being based on the amount of both suPAR and IL-6 in non-severely infected subjects.

The score may be a monotonically increasing function of the amount of suPAR and a monotonically increasing function of the amount of IL-6. In one embodiment, the function is linear.

In another embodiment, the score may be a decreasing function of the amount of suPAR and a decreasing function of the amount of IL-6. In this case, when the score is below a predetermined level, a severe infection is ruled in, the predetermined level being based on the amount of both suPAR and IL-6 in non-severely infected subjects.

The score may be a monotonically decreasing function of suPAR and a monotonically decreasing function of the amount of IL-6. In one embodiment, the function is linear.

In one embodiment, the score is based on the ratio of suPAR:IL-6.

In still another embodiment, the score is based on the ratio of IL-6:suPAR.

The predetermined level of any of the aspects of the present invention may be a reference value derived from population studies, including without limitation, such subjects having a known infection, subject having the same or similar age range, subjects in the same or similar ethnic group, or relative to the starting sample of a subject undergoing treatment for an infection. Such reference values can be derived from statistical analyses and/or risk prediction data of populations obtained from mathematical algorithms and computed indices of infection. Reference determinant indices can also be constructed and used using algorithms and other methods of statistical and structural classification.

In one embodiment of the present invention, the predetermined level is the amount (i.e. level) of (or a function of the amount of) IP-10 (and/or IL-6) and suPAR in a control sample derived from one or more subjects who do not have an infection (i.e., healthy, and or non-infectious individuals) or who do not have a severe infection (i.e., subjects who have a non-severe infection).

Generating scores (i.e. construction of clinical algorithms) may be carried out using methods known in the art and are discussed in detail below.

According to a specific embodiment, the above disclosed protein levels (either alone, as single markers or in combinations) are used to provide a risk assessment of the subject.

The term “risk assessment” refers to as assignment of a probability to experience certain adverse events (e.g. death, hospitalization or admission to ICU) to an individual. Hereby, the individual may preferably be accounted to a certain risk category, wherein categories comprise for instance high risk versus low risk, or risk categories based on numeral values, such as risk category 1, 2, 3, etc.

The risk assessment may be made in the hospital, for example in the emergency department of a hospital and may be part of a triaging of the subject. On the basis of the expression level of at least one of the above disclosed proteins, a decision may be made on which patient to attend to first.

Emergency departments (ED) are progressively overwhelmed by patients with both urgent and non-urgent problems. This leads to overfilled ED waiting rooms with long waiting times, detrimental outcomes and unsatisfied patients. As a result, patients needing urgent care may not be treated in time, whereas patients with non-urgent problems may unnecessarily receive expensive and dispensable treatments. Time to effective treatment is among the key predictors for outcomes across different medical conditions. For these reasons, the present inventors propose expression analysis of the presently disclosed proteins in a risk stratification system in the ED as an initial triage of medical patients.

Thus for example, the proteins described herein (e.g. at least one of TSG-14, AGER, ANG-2 or ST2) may be used together with triage systems for patient and resources allocation such as Emergency Severity Index (ESI) or Canadian Triage Acuity Scale (CTAS).

In another embodiment, the risk assessment is made in the intensive care unit of a hospital.

The risk measurement may be used to determine a management course for the patient. The risk measurement may aid in selection of treatment priority and also site-of-care decisions (i.e. outpatient vs. inpatient management) and early identification and organization of post-acute care needs.

When a patient has been assessed as being at high risk, the management course is typically more aggressive than if he had not been assessed as being at high risk. Thus, treatment options such as mechanical ventilation, life support, catheterization, hemofiltration, invasive monitoring, sedation, intensive care admission, surgical intervention, drug of last resort and hospital admittance may be selected which may otherwise not have been considered the preferred method of treatment if the patient had not been assessed as being at high risk.

The risk analysis may be carried out together with at least two, at least three, at least four, at least five, at least six, at least seven, at least eight, at least nine or at least nine parameters of a clinical index of the subject and providing a risk score based on the clinical index.

In one embodiment, the risk analysis is carried out together with all the parameters of a clinical index of the subject.

Exemplary clinical indices include but are not limited to Acute Physiology and Chronic Health Evaluation (APACHE II) as a measure of how likely to make it out of intensive care unit; Simplified Acute Physiology (SAP) score; Glasgow Coma Score (GCS) as an assessment of consciousness; Sequential Organ Failure Assessment (SOFA) score as an assessment of person's organ function or rate of failure; qSOFA (Quick SOFA) Score for Sepsis—identifies high-risk patients for in-hospital mortality with suspected infection outside the ICU; CURB-65 Score for Pneumonia Severity—estimates mortality of community-acquired pneumonia to help determine inpatient vs. outpatient treatment; National Early Warning Score (NEWS)—determines the degree of illness of a patient and prompts critical care intervention; Modified Early Warning Score (MEWS) for Clinical Deterioration—determines the degree of illness of a patient National Early Warning Score (NEWS) 2—Determines the degree of illness of a patient and prompts critical care intervention and; Apgar Assessment of a newborn's adjustment to life; Pain perception profile; visual analogue scale (VAS); quality of life metrics such as EDLQ, SF36; depression scale such as CES-D; impact of event scale (IES); or thrombosis risk assessment, or trend therein, or combination of above.

According to one embodiment, the clinical index is NEWS, NEWS 2 and MEWS.

According to a particular embodiment, the clinical index is Acute Physiology and Chronic Health Evaluation II (APACHE II). This system is an example of a severity of disease classification system that uses a point score based upon initial values of 12 routine physiologic measurements that include: temperature, mean arterial pressure, pH arterial, heart rate, respiratory rate, AaDO2 or PaO2, sodium, potassium, creatinine, hematocrit, white blood cell count, and Glasgow Coma Scale. These parameters are measured during the first 24 hours after admission, and utilized in additional to information about previous health status (recent surgery, history of severe organ insufficiency, immunocompromised state) and baseline demographics such as age.

An integer score from 0 to 71 is calculated wherein higher scores correspond to more severe disease and a higher risk of death.

Many other predictive models have been developed for various purposes which are contemplated by the present invention. Such predictive models are used for determining population-based outcome risks. By way of illustration and not as a limitation, a partial list of predictive models comprises SAPS II expanded and predicted mortality, SAPS II and predicted mortality, APACHE I-IV and predicted mortality, SOFA (Sequential Organ Failure Assessment), MODS (Multiple Organ Dysfunction Score), ODIN (Organ Dysfunctions and/or Infection), MPM (Mortality Probability Model), MPM II LODS (Logistic Organ Dysfunction System), TRIOS (Three days Recalibrated ICU Outcome Score), EUROSCORE (cardiac surgery), ONTARIO (cardiac surgery), Parsonnet score (cardiac-surgery), System 97 score (cardiac surgery), QMMI score (coronary surgery), Early mortality risk in redocoronary artery surgery, MPM for cancer patients, POSSUM (Physiologic and Operative Severity Score for the enUmeration of Mortality and Morbidity) (surgery, any), Portsmouth POSSUM (surgery, any), IRISS score: graft failure after lung transplantation, Glasgow Coma Score, ISS (Injury Severity Score), RTS (Revised Trauma Score), TRISS (Trauma Injury Severity Score), ASCOT (A Severity Characterization Of Trauma), 24 h-ICU Trauma Score, TISS (Therapeutic Intervention Scoring System), TISS-28 (simplified TISS), PRISM (Pediatric RISk of Mortality), P-MODS (Pediatric Multiple Organ Dysfunction Score), DORA (Dynamic Objective Risk Assessment), PELOD (Pediatric Logistic Organ Dysfunction), PIM II (Paediatric Index of Mortality II), PIM (Paediatric Index of Mortality), CRIB II (Clinical Risk Index for Babies), CRIB (Clinical Risk Index for Babies), SNAP (Score for Neonatal Acute Physiology), SNAP-PE (SNAP Perinatal Extension), SNAP II and SNAPPE II, MSSS (Meningococcal Septic Shock Score), GMSPS (Glasgow Meningococcal Septicaemia Prognostic Score), Rotterdam Score (meningococcal septic shock), Children's Coma Score (Raimondi), Paediatric Coma Scale (Simpson & Reilly), and Pediatric Trauma Score, Rochester criteria, Philadelphia Criteria, Milwaukee criteria, the last three being specific to neonatal fever/sepsis. Of course, the above list of quality of care metrics directed to health risk to the patient is not limiting, and other miscellaneous scores and assessments known in the medical field can be used.

Classification of subjects into subgroups (e.g. severe/non-severe; high risk, low risk etc.) as performed in aspects of the present invention is preferably done with an acceptable level of clinical or diagnostic accuracy. An “acceptable degree of diagnostic accuracy”, is herein defined as a test or assay (such as the test used in some aspects of the invention) in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.75, 0.80, desirably at least 0.85, more desirably at least 0.875, preferably at least 0.90, more preferably at least 0.925, and most preferably at least 0.95.

Alternatively, the methods may be used to rule in or rule out severity with at least 75% total accuracy, more preferably 80%, 85%, 90%, 95%, 97%, 98%, 99% or greater total accuracy.

Alternatively, the methods predict the correct management or treatment with an MCC larger than 0.2, 0.3, 0.4, 0.5, 0.6, 0.7, 0.8, 0.9 or 1.0.

On the basis of the classification of the infection, clinical decisions may be made.

According to some embodiments of the invention, the method further comprises informing the subject of results of the classification.

As used herein the phrase “informing the subject” refers to advising the subject that based on the diagnosis the subject should seek a suitable treatment regimen.

Once the diagnosis is determined, the results can be recorded in the subject's medical file, which may assist in selecting a treatment regimen and/or determining prognosis of the subject.

Examples of clinical decisions that may be made in light of a severe classification include oxygen therapy, non-invasive ventilation, mechanical ventilation, invasive monitoring, last-resort drug, sedation, intensive care admission, admission to the step-down unit, surgical intervention, hospital admittance, anti-viral drug, antibiotic treatment, anti-viral regimen, anti-fungal drug, immune-globulin treatment, glucocorticoid therapy, extracorporeal membrane oxygenation, kidney replacement therapy.

An example of a clinical decision that may be made in light of a non-severe classification may be isolation.

The antiviral drug may be selected from the group consisting of Remdesivir, Ribavirin, Adefovir, Tenofovir, Acyclovir, Brivudin, Cidofovir, Fomivirsen, Foscarnet, Ganciclovir, Penciclovir, Amantadine, Rimantadine, Zanamivir, Molnupiravir, Paxlovid, Oseltamivir phosphate, Ivermectin, Interferon beta, Interferon alfa, Interferon lambda, Nitazoxanide, Hydroxychloroquine, Peramivir, Baloxavir marboxil, Entecavir, lamivudine and Telbivudine.

Also contemplated are plasma treatments from infected persons who survived and/or anti-HIV drugs such as lopinavir and ritonavir, as well as chloroquine.

Specific examples for drugs that are routinely used for the treatment of COVID-19 include, but are not limited to, Lopinavir/Ritonavir, Nucleoside analogues, Neuraminidase inhibitors, Remdesivir, polypeptide (EK1), abidol, RNA synthesis inhibitors (such as TDF, 3TC), anti-inflammatory drugs (such as hormones and other molecules), Monoclonal antibodies (Ixagevimab plus Cilgavimab (Evusheld), Adrecizumab, Procizumab, Tixagevimab plus cilgavimab (Evusheld)), Chinese traditional medicine, such ShuFengJieDu Capsules and Lianhuaqingwen Capsule, could be the drug treatment options for COVID19.

If a bacterial infection is ruled in, the subject may be treated with an antibiotic or other antibacterial agents.

As used herein, the term “antibiotic agent” refers to a group of chemical substances, isolated from natural sources or derived from antibiotic agents isolated from natural sources, having a capacity to inhibit growth of, or to destroy bacteria. Examples of antibiotic agents include, but are not limited to; Amikacin; Amoxicillin; Ampicillin; Azithromycin; Azlocillin; Aztreonam; Aztreonam; Carbenicillin; Cefaclor; Cefepime; Cefetamet; Cefinetazole; Cefixime; Cefonicid; Cefoperazone; Cefotaxime; Cefotetan; Cefoxitin; Cefpodoxime; Cefprozil; Cefsulodin; Ceftazidime; Ceftizoxime; Ceftriaxone; Cefuroxime; Cephalexin; Cephalothin; Cethromycin; Chloramphenicol; Cinoxacin; Ciprofloxacin; Clarithromycin; Clindamycin; Cloxacillin; Co-amoxiclavuanate; Dalbavancin; Daptomycin; Dicloxacillin; Doxycycline; Enoxacin; Erythromycin estolate; Erythromycin ethyl succinate; Erythromycin glucoheptonate; Erythromycin lactobionate; Erythromycin stearate; Erythromycin; Fidaxomicin; Fleroxacin; Gentamicin; Imipenem; Kanamycin; Lomefloxacin; Loracarbef; Methicillin; Metronidazole; Mezlocillin; Minocycline; Mupirocin; Nafcillin; Nalidixic acid; Netilmicin; Nitrofurantoin; Norfloxacin; Ofloxacin; Oxacillin; Penicillin G; Piperacillin; Retapamulin; Rifaxamin, Rifampin; Roxithromycin; Streptomycin; Sulfamethoxazole; Teicoplanin; Tetracycline; Ticarcillin; Tigecycline; Tobramycin; Trimethoprim; Vancomycin; combinations of Piperacillin and Tazobactam; and their various salts, acids, bases, and other derivatives. Anti-bacterial antibiotic agents include, but are not limited to, aminoglycosides, carbacephems, carbapenems, cephalosporins, cephamycins, fluoroquinolones, glycopeptides, lincosamides, macrolides, monobactams, penicillins, quinolones, sulfonamides, and tetracyclines.

Antibacterial agents also include antibacterial peptides. Examples include but are not limited to abaecin; andropin; apidaecins; bombinin; brevinins; buforin II; CAP18; cecropins; ceratotoxin; defensins; dermaseptin; dermcidin; drosomycin; esculentins; indolicidin; LL37; magainin; maximum H5; melittin; moricin; prophenin; protegrin; and or tachyplesins.

Once the classifications are made, additional tests may be made in order to corroborate the result or to further classify the infectious agent.

Examples of such tests include PCR analysis, sequencing analysis, viral culture, antibody or antigen testing.

A “subject” in the context of the present invention may be a mammal (e.g. human, dog, cat, horse, cow, sheep, pig or goat). According to another embodiment, the subject is a bird (e.g. chicken, turkey, duck or goose). According to a particular embodiment, the subject is a human. The subject may be male or female. The subject may be an adult (e.g. older than 18, 21, or 22 years or a child (e.g. younger than 18, 21 or 22 years). In another embodiment, the subject is an adolescent (between 12 and 21 years), an infant (29 days to less than 2 years of age) or a neonate (birth through the first 28 days of life). In still another embodiment, the subject is over 60, 70 or even 80.

The subject of this aspect of the present invention may have symptoms of an infection.

Exemplary symptoms include, but are not limited to fever, headache, cough, runny nose, chills, muscle aches, loss of taste and/or loss of smell.

According to a particular embodiment, measuring the determinants (i.e. proteins) described herein above is carried out no more than 24 hours following the start of symptoms, no more than 36 hours following the start of symptoms, no more than 48 hours following the start of symptoms, no more than 72 hours following the start of symptoms, no more than 96 hours following the start of symptoms, no more than 1 week following the start of symptoms, or no more than 2 weeks following the start of symptoms.

According to another embodiment, the subject is asymptomatic.

It will be appreciated, whether symptomatic or asymptomatic, the subject may or may not be contagious.

In one embodiment, the subject does not have a chronic non-infectious disease such as cancer, a chronic immune disorder or a chronic inflammatory disorder.

In another embodiment, the subject does not have a coronary disease.

According to one embodiment, the subject is suspected of suffering from (or is confirmed as having) SIRS without infection, sepsis, severe sepsis or septic shock.

In one embodiment, the subject is hospitalized.

In another embodiment, the subject is non-hospitalized.

For any of the aspects disclosed herein, the term “measuring” or “measurement,” or alternatively “detecting” or “detection,” means assessing the presence, absence, quantity or amount (which can be an effective amount) of the determinant within a clinical or subject-derived sample, including the derivation of qualitative or quantitative concentration levels of such determinants.

Methods of measuring the level of protein determinants are well known in the art and include, e.g., immunoassays based on antibodies to proteins, aptamers or molecular imprints.

Protein determinants can be detected in any suitable manner, but are typically detected by contacting a sample from the subject with an antibody, which binds the protein determinant and then detecting the presence or absence of a reaction product. The antibody may be monoclonal, polyclonal, chimeric, or a fragment of the foregoing, and the step of detecting the reaction product may be carried out with any suitable immunoassay.

In one embodiment, the antibody which specifically binds the determinant is attached (either directly or indirectly) to a signal producing label, including but not limited to a radioactive label, an enzymatic label, a hapten, a reporter dye or a fluorescent label.

Immunoassays carried out in accordance with some embodiments of the present invention may be homogeneous assays or heterogeneous assays. In a homogeneous assay the immunological reaction usually involves the specific antibody (e.g., anti-determinant antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels, which may be employed, include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate, pipette tip or slide, and contacted with the specimen suspected of containing the antigen in a liquid phase.

The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are oligonucleotides, immunoblotting, immunofluorescence methods, immunoprecipitation, chemiluminescence methods, electrochemiluminescence (ECL) or enzyme-linked immunoassays.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al., titled “Methods for Modulating Ligand-Receptor Interactions and their Application,” U.S. Pat. No. 4,659,678 to Forrest et al., titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.” The determinant can also be detected with antibodies using flow cytometry. Those skilled in the art will be familiar with flow cytometric techniques which may be useful in carrying out the methods disclosed herein (Shapiro 2005). These include, without limitation, Cytokine Bead Array (Becton Dickinson) and Luminex technology.

Antibodies can be conjugated to a solid support suitable for a diagnostic assay (e.g., beads such as magnetic beads, protein A or protein G agarose, microspheres, plates, slides, pipette tip or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

In particular embodiments, the antibodies of the present invention comprise monoclonal antibodies.

In other embodiments, the antibodies of the present invention comprise polyoclonal antibodies.

Suitable sources for antibodies for the detection of determinants include commercially available sources such as, for example, Abazyme, Abnova, AssayPro, Affinity Biologicals, AntibodyShop, Aviva bioscience, Biogenesis, Biosense Laboratories, Calbiochem, Cell Sciences, Chemicon International, Chemokine, Clontech, Cytolab, DAKO, Diagnostic BioSystems, eBioscience, Endocrine Technologies, Enzo Biochem, Eurogentec, Fusion Antibodies, Genesis Biotech, GloboZymes, Haematologic Technologies, Immunodetect, Immunodiagnostik, Immunometrics, Immunostar, Immunovision, Biogenex, Invitrogen, Jackson ImmunoResearch Laboratory, KMI Diagnostics, Koma Biotech, LabFrontier Life Science Institute, Lee Laboratories, Lifescreen, Maine Biotechnology Services, Mediclone, MicroPharm Ltd., ModiQuest, Molecular Innovations, Molecular Probes, Neoclone, Neuromics, New England Biolabs, Novocastra, Novus Biologicals, Oncogene Research Products, Orbigen, Oxford Biotechnology, Panvera, PerkinElmer Life Sciences, Pharmingen, Phoenix Pharmaceuticals, Pierce Chemical Company, Polymun Scientific, Polysiences, Inc., Promega Corporation, Proteogenix, Protos Immunoresearch, QED Biosciences, Inc., R&D Systems, Repligen, Research Diagnostics, Roboscreen, Santa Cruz Biotechnology, Seikagaku America, Serological Corporation, Serotec, SigmaAldrich, StemCell Technologies, Synaptic Systems GmbH, Technopharm, Terra Nova Biotechnology, TiterMax, Trillium Diagnostics, Upstate Biotechnology, US Biological, Vector Laboratories, Wako Pure Chemical Industries, and Zeptometrix. However, the skilled artisan can routinely make antibodies, against any of the polypeptide determinants described herein.

The presence of a label can be detected by inspection, or a detector which monitors a particular probe or probe combination is used to detect the detection reagent label. Typical detectors include spectrophotometers, phototubes and photodiodes, microscopes, scintillation counters, cameras, film and the like, as well as combinations thereof. Those skilled in the art will be familiar with numerous suitable detectors that widely available from a variety of commercial sources and may be useful for carrying out the method disclosed herein. Commonly, an optical image of a substrate comprising bound labeling moieties is digitized for subsequent computer analysis. See generally The Immunoassay Handbook [The Immunoassay Handbook. Third Edition. 2005].

Antibodies suitable for specifically detecting ST2 include Recombinant Rabbit anti-human monoclonal antibody to ST2 (ab259721) (Abcam), Mouse anti-human monoclonal antibody to ST2/IL-33R Antibody, Clone #97203, (MAB523) (Biotechne® R&D Systems), IL-33R (ST2) Mouse anti-human Monoclonal Antibody to ST2 (IL-33R), Clone hIL33Rcap, eBioscience™ Catalog #17-9338-42 (invitrogen).

Antibodies suitable for specifically detecting ANG-2 include Mouse anti-human monoclonal antibody to Angiopoietin-2, Clone #85816, (MAB098) (Biotechne® R&D Systems), Recombinant rabbit anti-human monoclonal antibody to Angiopoietin 2/ANG-2 (ab285368) (Abcam), Rabbit anti-human polyclonal antibody to Angiopoietin 2, Catalog #PA5-27297, (Invitrogen).

Antibodies suitable for specifically detecting AGER include Recombinant Rabbit anti-human monoclonal Antibody to AGER/RAGE (ab289826) (abeam); Mouse anti-human monoclonal antibody to AGER/RAGE, Clone #176902, (MAB11451) (Biotechne® R&D Systems) and Rabbit anti-human polyclonal Antibody to AGER/RAGE, (TA346145) (OriGene).

Antibodies suitable for specifically detecting TSG-14 include Recombinant Rabbit anti-human monoclonal Antibody to TSG-14/Pentraxin 3/PTX3 antibody (ab242624) (abeam); Mouse anti-human monoclonal antibody to TSG-14/Pentraxin 3, Clone #247911, (MAB1826) (Biotechne® R&D Systems); and Rabbit anti-human polyclonal Antibody to TSG-14/PTX3 (SAB4502545), (Sigma-Aldrich®).

Antibodies suitable for specifically detecting MR-proADM include Mouse anti-human monoclonal antibody to MR-Pro ADM, SAB4200700 (Sigma-Aldrich®) and Rabbit anti-human Polyclonal Antibody to Proadrenomedullin (45-92), (TA364336) (OriGene). Measurement of MR-proADM may be a proxy for measurement of Adrenomedullin (ADM). MR-proADM is a fragment of 48 amino acids which splits from proADM molecule in a 1:1 ratio with Adrenomedullin.

Antibodies suitable for specifically detection IL-6 include but are not limited to Mouse anti-human monoclonal antibody to IL-6 (MAB2063) (Biotechne® R&D Systems)., Mouse anti-human monoclonal antibody to IL-6, Clone 5IL6, Catalog #M620, (Invitrogen) and Mouse anti-human monoclonal antibody to IL-6, clone OTI3G9, (TA500067) (OriGene).

Antibodies suitable for detecting IL-10 include Recombinant Rabbit anti-human monoclonal Antibody to IL-10 (ab244835) (abcam); Mouse anti-human monoclonal antibody to IL-10, Clone #127107, (MAB2172) (Biotechne® R&D Systems); and Rat anti-human monoclonal antibody to IL-10, Clone JES3-9D7, eBioscience™, Catalog #14-7108-81 (Invitrogen).

Antibodies suitable for measuring TRAIL include without limitation: Mouse, Monoclonal (55B709-3) IgG (Thermo Fisher Scientific); Mouse, Monoclonal (2E5) IgG1 (Enzo Lifesciences); Mouse, Monoclonal (2E05) IgG1; Mouse, Monoclonal (M912292) IgG1 kappa (My BioSource); Mouse, Monoclonal (IIIF6) IgG2b; Mouse, Monoclonal (2E1-1B9) IgG1 (EpiGentek); Mouse, Monoclonal (RIK-2) IgG1, kappa (BioLegend); Mouse, Monoclonal M181 IgG1 (Immunex Corporation); Mouse, Monoclonal VI10E IgG2b (Novus Biologicals); Mouse, Monoclonal MAB375 IgG1 (R&D Systems); Mouse, Monoclonal MAB687 IgG1 (R&D Systems); Mouse, Monoclonal HS501 IgG1 (Enzo Lifesciences); Mouse, Monoclonal clone 75411.11 Mouse IgG1 (Abcam); Mouse, Monoclonal T8175-50 IgG (X-Zell Biotech Co); Mouse, Monoclonal 2B2.108 IgG1; Mouse, Monoclonal B-T24 IgG1 (Cell Sciences); Mouse, Monoclonal 55B709.3 IgG1 (Thermo Fisher Scientific); Mouse, Monoclonal D3 IgG1 (Thermo Fisher Scientific); Goat, Polyclonal C19 IgG; Rabbit, Polyclonal H257 IgG (Santa Cruz Biotechnology); Mouse, Monoclonal 500-M49 IgG; Mouse, Monoclonal 05-607 IgG; Mouse, Monoclonal B-T24 IgG1 (Thermo Fisher Scientific); Rat, Monoclonal (N2B2), IgG2a, kappa (Thermo Fisher Scientific); Mouse, Monoclonal (1A7-2B7), IgG1 (Genxbio); Mouse, Monoclonal (55B709.3), IgG (Thermo Fisher Scientific); Mouse, Monoclonal B-S23*IgG1 (Cell Sciences), Human TRAIL/TNFSF10 MAb (Clone 75411), Mouse IgG1 (R&D Systems); Human TRAIL/TNFSF10 MAb (Clone 124723), Mouse IgG1 (R&D Systems) and Human TRAIL/TNFSF10 MAb (Clone 75402), Mouse IgG1 (R&D Systems).

Antibodies suitable for measuring IP-10 include without limitation: Mouse anti-human CXCL10 (IP-10) Monoclonal Antibody (Cat. No. 524401) (BioLegend), Rabbit anti-human CXCL10 (IP-10) polyclonal Antibody (ab9807) (Abeam), Mouse anti-human CXCL10 (IP-10) Monoclonal Antibody (4D5) (MCA1693) (Bio-Rad), Goat anti-human CXCL10 (IP-10) Monoclonal Antibody (PA5-46999) (Invitrogen), Mouse anti-human CXCL10 (IP-10) Monoclonal Antibody (MA5-23819) (Invitrogen).

Antibodies suitable for measuring CRP include without limitation: Rabbit anti-Human C-Reactive Protein/CRP polyclonal antibody (ab31156) (Abcam), Sheep anti-Human C-Reactive Protein/CRP Polyclonal antibody (AF1707) (R&D Systems), rabbit anti-Human C-Reactive Protein/CRP Polyclonal antibody (C3527) (Sigma-Aldrich), Mouse anti-Human C-Reactive Protein/CRP monoclonal antibody (C1688) (MilliporeSigma).

Continuous and discontinuous epitopes present in the protein suPAR and its cleavage products may be used to monitor their presence and abundance in a biological fluid by immunodetection with mono- or polyclonal antibodies. Antibodies directed to accessible epitopes common to suPAR and its cleavage products (e.g. D2D3) can be used to detect both suPAR and its cleavage products in a biological fluid. Since there is a one-to-one relationship between suPAR and its cleavage products, an antibody that is directed to an epitope that is common to both full length suPAR and, say, the D2D3 cleavage product will at the same time directly and indirectly measure the suPAR level. That is to say, a value of, say, 3 ng/ml as measured in the assay is regarded as indicating a suPAR level of 3 ng/ml, even though some of the protein that was detected may have been the D2D3 cleavage product. In the context of the assay, therefore, “suPAR” refers to full length suPAR and its cleavage product D2D3. The term D2D3 is used to denote any suPAR-derived fragment corresponding to the 84-277 region of suPAR and having an N-terminus lying in the 84-92 amino acid region of suPAR and a C-terminus corresponding to the C-terminus of suPAR (amino acid 277), for example 84-277, 88-277, 90-277 and 92-277. suPAR levels may be measured in body fluids by the methods taught in WO 2008/077958, the contents of which are incorporated herein by reference.

More specifically, suPAR levels may be determined by ELISA assay as follows: Nunc Maxisorp ELISA-plates (Nunc, Roskilde, Denmark) are coated overnight at 4° C. with a monoclonal rat anti-suPAR antibody (VG-1, ViroGates A/S, Copenhagen, Denmark, 3 μg/ml, 100 mt/well). Plates are blocked with PBS buffer+1% BSA and 0.1% Tween 20, 1 hour at room temperature, and washed 3 times with PBS buffer containing 0.1% Tween 20. 85 mi dilution buffer (100 mm phosphate, 97.5 mm NaCl, 10 g L¹ bovine serum albumin (BSA, Fraction V, Roche Diagnostics GmbH Penzberg, Germany), 50 U mL 1 heparin sodium salt (Sigma Chemical Co., St. Louis, MO), 0.1% (v/v) Tween 20, pH 7.4) containing 1.5 μg/ml HRP labeled mouse anti-suPAR antibody (VG-2-HR-P, ViroGates) and 15 mi plasma (or serum or urine) sample is added in duplicates to the ELISA plate. After 1 hour of incubation at 37° C., plates are washed 10 times with PBS buffer+0.1% Tween 20 and 100 mt/well HRP substrate added (Substrate Reagent Pack, R&D Systems Minneapolis, Minnesota). The colour reaction is stopped after 30 min using 50 mi per well IM H2SO4 and measured at 450 nm.

Furthermore, suPAR can be measured in bodily fluids using commercially available CE/IVD approved assays such as the suPARnostic© product line according to the manufacturer's instructions. In the TRIAGE II trials, suPAR was quantified using the suPARnostic Quick Triage lateral flow assay.

The suPAR level may, for example, be assayed using the suPARnostic@Autoflex ELISA test sold by ViroGates A/S, Banevaenget 13, DK-3460 Birkerpd, Denmark. Alternatively, suPAR levels can be measured by proteomic approaches such as western blot, Luminex, MALDI-TOF, HPLC or Genspeed device and automated immune analyzer platforms such as Bayer Centaur, Abbott Architect, Abbott AxSym, Roche CO BAS and the Axis Shield Afinion or using turbidimetric assays such as suPARnostic® Turbilatex on Roche, Cobas cIII, Cobas c501/2+c701/2, or Siemens ADVIA XPT or Centaur or Abbott Architect.

The suPAR level in blood may be measured directly in a blood sample or in serum, plasma or urine. Anticoagulant plasma is preferred e.g. EDTA or Citrate plasma. Where the biological sample is urine, the measurements may be based on the urine suPAR/creatinine value from a subject, since this value is known to be highly correlated to the concentration of suPAR in a plasma sample derived from the same subject. Thus, urine samples may also be employed for the measurement of suPAR, where the measured level in urine is normalized for protein content (e.g. using creatinine). These normalized values may be employed as a marker for the purposes of the present invention.

A “sample” in the context of the present invention is a biological sample isolated from a subject and can include, by way of example and not limitation, whole blood, serum, plasma, saliva, mucus, breath, urine, CSF, sputum, sweat, stool, hair, seminal fluid, biopsy, rhinorrhea, tissue biopsy, cytological sample, platelets, reticulocytes, leukocytes, epithelial cells, or whole blood cells.

In a particular embodiment, the sample is a blood sample—e.g. serum, plasma, or whole blood. The sample may be a venous sample, peripheral blood mononuclear cell sample or a peripheral blood sample. In one embodiment, the sample comprises white blood cells including for example granulocytes, lymphocytes and/or monocytes. In one embodiment, the sample is depleted of red blood cells.

The subject is typically suffering from a bacterial or viral infection.

The bacterial or viral infection may be an acute or chronic infection.

A chronic infection is an infection that develops slowly and lasts a long time. Viruses that may cause a chronic infection include Hepatitis C and HIV. One difference between acute and chronic infection is that during acute infection the immune systemoftenproduces IgM+ antibodies against the infectious agent, whereas the chronic phase of the infection is usually characteristic of IgM−/IgG+ antibodies. In addition, acute infections cause immune mediated necrotic processes while chronic infections often cause inflammatory mediated fibrotic processes and scaring (e.g. Hepatitis C in the liver). Thus, acute and chronic infections may elicit different underlying immunological mechanisms.

According to a particular embodiment, the infection that is diagnosed is an acute infection.

Exemplary viral diseases which may be diagnosed according to the methods described herein are summarized in Table 2.

TABLE 2
Diseases

gastroenteritis
keratoconjunctivitis
pharyngitis
croup
pharyngoconjunctival fever
pneumonia
cystitis
Hand, foot and mouth disease
pleurodynia
aseptic meningitis
pericarditis
myocarditis
infectious mononucleosis
Burkitt's lymphoma
Hodgkin's lymphoma
nasopharyngeal carcinoma
acute hepatitis
chronic hepatitis
hepatic cirrhosis
hepatocellular carcinoma
herpes labialis, cold sores - can recur by latency
gingivostomatitis in children
tonsillitis & pharyngitis in adults
keratoconjunctivitis
Aseptic meningitis
infectious mononucleosis
Cytomegalic inclusion disease
Kaposi sarcoma
multicentric Castleman disease
primary effusion lymphoma
AIDS
influenza
(Reye syndrome)
measles
postinfectious encephalomyelitis
mumps
hyperplastic epithelial lesions (common, flat, plantar and anogenital warts, laryngeal
papillomas, epidermodysplasia verruciformis
Malignancies for some species (cervical carcinoma, squamous cell carcinomas)
croup
pneumonia
bronchiolitis
common cold[
poliomyelitis
rabies (fatal encephalitis)
congenital rubella
German measles
chickenpox
herpes zoster
Congenital varicella syndrome

According to a specific embodiment, the viral disease is COVID-19.

Exemplary virus-causing families are summarized in Table 3, herein below.

TABLE 3

| | Baltimore | | |
|---|---|---|---|
| Family | group | Important species | envelopment |
| Adenoviridae | Group I | Adenovirus | non- |
| | (dsDNA) | | enveloped |
| Herpesviridae | Group I | Herpes simplex, type 1, Herpes simplex, type | enveloped |
| | (dsDNA) | 2, Varicella-zoster virus, Epstein-Barr virus, | |
| | | Human cytomegalovirus, Human herpesvirus, | |
| | | type 8 | |
| Papillomaviridae | Group I | Human papillomavirus | non- |
| | (dsDNA) | | enveloped |
| Polyomaviridae | Group I | BK virus, JC virus | non- |
| | (dsDNA) | | enveloped |
| Poxviridae | Group I | Smallpox | enveloped |
| | (dsDNA) | | |
| Hepadnaviridae | Group VII | Hepatitis B virus | enveloped |
| | (dsDNA-RT) | | |
| Parvoviridae | Group II | Parvovirus B19 | non- |
| | (ssDNA) | | enveloped |
| Astroviridae | Group IV | Human astrovirus | non- |
| | (positive-sense | | enveloped |
| | SSRNA) | | |
| Caliciviridae | Group IV | Norwalk virus | non- |
| | (positive-sense | | enveloped |
| | ssRNA) | | |
| Picornaviridae | Group IV | coxsackievirus, hepatitis A virus, poliovirus, | non- |
| | (positive-sense | rhinovirus | enveloped |
| | ssRNA) | | |
| Coronaviridae | Group IV | Severe acute respiratory syndrome virus | enveloped |
| | (positive-sense | | |
| | ssRNA) | | |
| Flaviviridae | Group IV | Hepatitis C virus, yellow fever virus, dengue | enveloped |
| | (positive-sense | virus, West Nile virus, TBE virus | |
| | SSRNA) | | |
| Togaviridae | Group IV | Rubella virus | enveloped |
| | (positive-sense | | |
| | SSRNA) | | |
| Hepeviridae | Group IV | Hepatitis E virus | non- |
| | (positive-sense | | enveloped |
| | SSRNA) | | |
| Retroviridae | Group VI | Human immunodeficiency virus (HIV) | enveloped |
| | (ssRNA-RT) | | |
| Orthomyxoviridae | (negative- | Influenza virus | enveloped |
| | Group V | | |
| | sense ssRNA) | | |
| Arenaviridae | Group V | Lassa virus | enveloped |
| | (negative- | | |
| | sense ssRNA) | | |
| Bunyaviridae | Group V | Crimean-Congo hemorrhagic fever virus, | enveloped |
| | (negative- | Hantaan virus | |
| | sense ssRNA) | | |
| Filoviridae | Group V | Ebola virus, Marburg virus | enveloped |
| | (negative- | | |
| | sense ssRNA) | | |
| Paramyxoviridae | Group V | Measles virus, Mumps virus, Parainfluenza | enveloped |
| | (negative- | virus, Respiratory syncytial virus, | |
| | sense ssRNA) | | |
| Rhabdoviridae | Group V | Rabies virus | enveloped |
| | (negative- | | |
| | sense ssRNA) | | |
| Unassigned | Group V | Hepatitis D | enveloped |
| | (negative- | | |
| | sense ssRNA) | | |
| Reoviridae | Group III | Rotavirus, Orbivirus, Coltivirus, Banna virus | non- |
| | (dsRNA) | | enveloped |

According to another specific embodiment, the virus is Human metapneumovirus, Bocavirus or Enterovirus.

According to another specific embodiment, the virus is RSV, Flu A, Flu B, HCoV or SARS-Cov-2.

Examples of coronaviruses include: human coronavirus 229E, human coronavirus OC43, SARS-CoV, HCoV NL63, HKU1, MERS-CoV and SARS-CoV-2.

According to a particular embodiment, the coronavirus is SARS-CoV-2.

Bacterial infections which may be ruled in according to embodiments of the invention may be the result of gram-positive, gram-negative bacteria or atypical bacteria.

The term “Gram-positive bacteria” refers to bacteria that are stained dark blue by Gram staining. Gram-positive organisms are able to retain the crystal violet stain because of the high amount of peptidoglycan in the cell wall.

The term “Gram-negative bacteria” refers to bacteria that do not retain the crystal violet dye in the Gram staining protocol.

The term “Atypical bacteria” are bacteria that do not fall into one of the classical “Gram” groups. They are usually, though not always, intracellular bacterial pathogens. They include, without limitations, Mycoplasmas spp., Legionella spp. Rickettsiae spp., and Chlamydiae spp.

The present inventors have further uncovered a unique set of immune proteins, found to be specific markers for viral or bacterial infections, markers of infection in general and/or markers of severity.

Thus, according to another aspect of the present invention, there is provided a method of distinguishing between a viral and bacterial infection in a subject comprising:
(a) measuring an expression level of a combination of proteins, the combination being set forth in Groups 1 or 2; and
(b) determining whether the infection is bacterial or viral based on the expression level.

According to still another aspect of the invention there is provided method of diagnosing an infectious disease in a subject comprising:
(a) measuring an expression level of a combination of proteins, the combination being set forth in Groups 5 or 6; and
(b) determining whether the disease is infectious or non-infectious based on the expression level.

According to another aspect of the invention there is provided a method of determining the severity of an infectious disease in a subject comprising:
(a) measuring expression level of a combination of proteins, the combination being set forth in Groups 3 or 4; and
(b) determining the severity based on the expression level.

The concentrations of each of the above identified polypeptides may be combined (e.g. by way of a pre-determined mathematical function) to compute a score and the score may be compared to a predetermined reference value as further described herein below.

The markers in each of the combinations set forth in Groups 1 or 2 are either upregulated or down-regulated in bacterial disease (as compared to healthy patients or virally infected patients).

The direction which the markers move is summarized in Table 5.

The markers in each of the combinations set forth in Groups 3 or 4 are either upregulated or down-regulated in severe disease (as compared to non-severely infected patients). The direction which the markers move is presented in Table 6.

The markers in each of the combinations set forth in Groups 5 and 6 are either upregulated or down-regulated in infectious disease (as compared to healthy patients). The direction which the markers move is presented in Table 7.

For classifying a viral disease, in one aspect at least one protein in Table 5 is measured and at least one protein in Table 6 is measured.

In one embodiment, the classification is carried out by generating a score based on the amount of the proteins listed in the combinations set forth in Group 1, 2, 3, 4, 5 or 6 Particular contemplated combinations set forth in Group 1 include PLA2G2A and TRAIL and IP-10 and CRP; RNASE3 and TRAIL and IP-10 and CRP; TGFA and TRAIL and IP-10 and CRP; AZU1 and TRAIL and IP-10 and CRP; CD177 and TRAIL and IP-10 and CRP; CLEC4D and TRAIL and IP-10 and CRP; CEACAM8 and TRAIL and IP-10 and CRP; HGF and TRAIL and IP-10 and CRP; VWA1 and TRAIL and IP-10 and CRP; PRTN3 and TRAIL and IP-10 and CRP; MMP9 and TRAIL and IP-10 and CRP; GH2 and TRAIL and IP-10 and CRP; LCN2 and TRAIL and IP-10 and CRP; CST7 and TRAIL and IP-10 and CRP; EPO and TRAIL and IP-10 and CRP; DEFA1_DEFAIB and TRAIL and IP-10 and CRP; LBP and TRAIL and IP-10 and CRP; OLR1 and TRAIL and IP-10 and CRP; LRIG1 and TRAIL and IP-10 and CRP; VSTM1 and TRAIL and IP-10 and CRP; TNFRSF10C and TRAIL and IP-10 and CRP; JCHAIN and TRAIL and IP-10 and CRP; C4BPB and TRAIL and IP-10 and CRP; MPO and TRAIL and IP-10 and CRP; TNFSF14 and TRAIL and IP-10 and CRP; QPCT and TRAIL and IP-10 and CRP; IL1B and TRAIL and IP-10 and CRP; ST6GAL1 and TRAIL and IP-10 and CRP; PGLYRP1 and TRAIL and IP-10 and CRP; PIGR and TRAIL and IP-10 and CRP; RARRES2 and TRAIL and IP-10 and CRP; LIF and TRAIL and IP-10 and CRP; CCL23 and TRAIL and IP-10 and CRP; SPARC and TRAIL and IP-10 and CRP; FCAR and TRAIL and IP-10 and CRP; VEGFA and TRAIL and IP-10 and CRP; VNN2 and TRAIL and IP-10 and CRP; CCL18 and TRAIL and IP-10 and CRP; CA4 and TRAIL and IP-10 and CRP; TIE1 and TRAIL and IP-10 and CRP; MCFD2 and TRAIL and IP-10 and CRP; TGFB1 and TRAIL and IP-10 and CRP; C2 and TRAIL and IP-10 and CRP; PADI2 and TRAIL and IP-10 and CRP; NID1 and TRAIL and IP-10 and CRP; ERP44 and TRAIL and IP-10 and CRP; CD34 and TRAIL and IP-10 and CRP; NAAA and TRAIL and IP-10 and CRP; PRTG and TRAIL and IP-10 and CRP; TPPP3 and TRAIL and IP-10 and CRP; SEZ6L and TRAIL and IP-10 and CRP; CPM and TRAIL and IP-10 and CRP; MEGF10 and TRAIL and IP-10 and CRP; GDF2 and TRAIL and IP-10 and CRP; MCAM and TRAIL and IP-10 and CRP; ICOSLG and TRAIL and IP-10 and CRP; AOC3 and TRAIL and IP-10 and CRP; NCAN and TRAIL and IP-10 and CRP; CCL25 and TRAIL and IP-10 and CRP; IL22RA1 and TRAIL and IP-10 and CRP; HSD11B1 and TRAIL and IP-10 and CRP; APLP1 and TRAIL and IP-10 and CRP; CTSV and TRAIL and IP-10 and CRP; LAG3 and TRAIL and IP-10 and CRP; DKK3 and TRAIL and IP-10 and CRP; ITM2A and TRAIL and IP-10 and CRP; SCGB1A1 and TRAIL and IP-10 and CRP; LGALS4 and TRAIL and IP-10 and CRP; EPCAM and TRAIL and IP-10 and CRP; TNFSF11 and TRAIL and IP-10 and CRP; RBP2 and TRAIL and IP-10 and CRP; GPA33 and TRAIL and IP-10 and CRP; FABP1 and TRAIL and IP-10 and CRP; FGF23 and TRAIL and IP-10 and CRP; REG1B and TRAIL and IP-10 and CRP; REG1A and TRAIL and IP-10 and CRP; MMP12 and TRAIL and IP-10 and CRP; CHI3L1 and TRAIL and IP-10 and CRP; ULBP2 and TRAIL and IP-10 and CRP; PRL and TRAIL and IP-10 and CRP; DSC2 and TRAIL and IP-10 and CRP; CD300LG and TRAIL and IP-10 and CRP; TNFRSF9 and TRAIL and IP-10 and CRP; ROR1 and TRAIL and IP-10 and CRP; CCL4 and TRAIL and IP-10 and CRP; CD300LF and TRAIL and IP-10 and CRP; IL4R and TRAIL and IP-10 and CRP; IL17A and TRAIL and IP-10 and CRP; TNFRSF1A and TRAIL and IP-10 and CRP; CDON and TRAIL and IP-10 and CRP; CCN4 and TRAIL and IP-10 and CRP; TNFRSF4 and TRAIL and IP-10 and CRP; MMP13 and TRAIL and IP-10 and CRP; AKT1S1 and TRAIL and IP-10 and CRP; SCARF2 and TRAIL and IP-10 and CRP; VTA1 and TRAIL and IP-10 and CRP; TRAF2 and TRAIL and IP-10 and CRP; USP8 and TRAIL and IP-10 and CRP; HSPG2 and TRAIL and IP-10 and CRP; SIT1 and TRAIL and IP-10 and CRP; TSHB and TRAIL and IP-10 and CRP; ANGPT2 and TRAIL and IP-10 and CRP; CD163 and TRAIL and IP-10 and CRP; FLRT2 and TRAIL and IP-10 and CRP; CDH6 and TRAIL and IP-10 and CRP; TIMD4 and TRAIL and IP-10 and CRP; CD93 and TRAIL and IP-10 and CRP; CLEC10A and TRAIL and IP-10 and CRP; PRKRA and TRAIL and IP-10 and CRP; CCL14 and TRAIL and IP-10 and CRP; COMP and TRAIL and IP-10 and CRP; THBS4 and TRAIL and IP-10 and CRP; NRP1 and TRAIL and IP-10 and CRP; TIMP1 and TRAIL and IP-10 and CRP; MAP4K5 and TRAIL and IP-10 and CRP; ITGB2 and TRAIL and IP-10 and CRP; AMIGO2 and TRAIL and IP-10 and CRP; ADAM15 and TRAIL and IP-10 and CRP; AXL and TRAIL and IP-10 and CRP; ESAM and TRAIL and IP-10 and CRP; CD6 and TRAIL and IP-10 and CRP; CD55 and TRAIL and IP-10 and CRP; TNXB and TRAIL and IP-10 and CRP; GPNMB and TRAIL and IP-10 and CRP; SELE and TRAIL and IP-10 and CRP; IL10RB and TRAIL and IP-10 and CRP; ADAM8 and TRAIL and IP-10 and CRP; CDH5 and TRAIL and IP-10 and CRP; VASN and TRAIL and IP-10 and CRP; COL1A1 and TRAIL and IP-10 and CRP; ROBO1 and TRAIL and IP-10 and CRP; SCARF1 and TRAIL and IP-10 and CRP; ICAM1 and TRAIL and IP-10 and CRP; SELP and TRAIL and IP-10 and CRP; FCGR3B and TRAIL and IP-10 and CRP; OSCAR and TRAIL and IP-10 and CRP; BOC and TRAIL and IP-10 and CRP; PTPRM and TRAIL and IP-10 and CRP; GFRA2 and TRAIL and IP-10 and CRP; EDIL3 and TRAIL and IP-10 and CRP; APOH and TRAIL and IP-10 and CRP; ALCAM and TRAIL and IP-10 and CRP; DDR1 and TRAIL and IP-10 and CRP; HYOU1 and TRAIL and IP-10 and CRP; SIGLEC7 and TRAIL and IP-10 and CRP; ARID4B and TRAIL and IP-10 and CRP; CNTN3 and TRAIL and IP-10 and CRP; ENTPD6 and TRAIL and IP-10 and CRP; ARSA and TRAIL and IP-10 and CRP; CDH2 and TRAIL and IP-10 and CRP; B4GAT1 and TRAIL and IP-10 and CRP; IFNLR1 and TRAIL and IP-10 and CRP; PLXNB2 and TRAIL and IP-10 and CRP; ERBB3 and TRAIL and IP-10 and CRP; GLB1 and TRAIL and IP-10 and CRP; IRAG2 and TRAIL and IP-10 and CRP; PCSK9 and TRAIL and IP-10 and CRP; CASP1 and TRAIL and IP-10 and CRP; SSB and TRAIL and IP-10 and CRP; FOSB and TRAIL and IP-10 and CRP; GRN and TRAIL and IP-10 and CRP; ACP5 and TRAIL and IP-10 and CRP; UXS1 and TRAIL and IP-10 and CRP; SLC27A4 and TRAIL and IP-10 and CRP; INHBC and TRAIL and IP-10 and CRP; BID and TRAIL and IP-10 and CRP; ANGPTL1 and TRAIL and IP-10 and CRP; TPP1 and TRAIL and IP-10 and CRP; DNAJB8 and TRAIL and IP-10 and CRP; CBLN4 and TRAIL and IP-10 and CRP; ADAMTS13 and TRAIL and IP-10 and CRP; TACC3 and TRAIL and IP-10 and CRP; TXNRD1 and TRAIL and IP-10 and CRP; SMPDL3A and TRAIL and IP-10 and CRP; SMPD1 and TRAIL and IP-10 and CRP; GOLM2 and TRAIL and IP-10 and CRP; CDKN1A and TRAIL and IP-10 and CRP; C2CD2L and TRAIL and IP-10 and CRP; ST3GAL1 and TRAIL and IP-10 and CRP; GGH and TRAIL and IP-10 and CRP; JUN and TRAIL and IP-10 and CRP; AIF1 and TRAIL and IP-10 and CRP; WARS and TRAIL and IP-10 and CRP; GBP4 and TRAIL and IP-10 and CRP; TINAGLI and TRAIL and IP-10 and CRP; LGALS9 and TRAIL and IP-10 and CRP; EZR and TRAIL and IP-10 and CRP; TCN2 and TRAIL and IP-10 and CRP; SCRN1 and TRAIL and IP-10 and CRP; PRDX1 and TRAIL and IP-10 and CRP; CTSC and TRAIL and IP-10 and CRP; LAMP3 and TRAIL and IP-10 and CRP; MSTN and TRAIL and IP-10 and CRP; TCL1A and TRAIL and IP-10 and CRP; SFTPA2 and TRAIL and IP-10 and CRP; CCL8 and TRAIL and IP-10 and CRP; SAMD9L and TRAIL and IP-10 and CRP; TRIM21 and TRAIL and IP-10 and CRP; AGER and TRAIL and IP-10 and CRP; NADK and TRAIL and IP-10 and CRP; TYMP and TRAIL and IP-10 and CRP; LAP3 and TRAIL and IP-10 and CRP; AGR2 and TRAIL and IP-10 and CRP; CCL7 and TRAIL and IP-10 and CRP; RRM2 and TRAIL and IP-10 and CRP; BRK1 and TRAIL and IP-10 and CRP; DDX58 and TRAIL and IP-10 and CRP; CXCL11 and TRAIL and IP-10 and CRP; KRT19 and TRAIL and IP-10 and CRP.

Particular combinations set forth in Group 2 include PLA2G2A and FGF23; PLA2G2A and CCL20; PLA2G2A and EPO; PLA2G2A and REG1B; PLA2G2A and REG1A; PLA2G2A and CTSB; PLA2G2A and MMP12; PLA2G2A and CHI3L1; PLA2G2A and ULBP2; PLA2G2A and PRL; CSF3 and FGF23; CSF3 and CCL20; CSF3 and EPO; CSF3 and REG1B; CSF3 and REG1A; CSF3 and CTSB; CSF3 and MMP12; CSF3 and CHI3L1; CSF3 and ULBP2; CSF3 and PRL; MMP8 and FGF23; MMP8 and CCL20; MMP8 and EPO; MMP8 and REG1B; MMP8 and REG1A; MMP8 and CTSB; MMP8 and MMP12; MMP8 and CHI3L1; MMP8 and ULBP2; MMP8 and PRL; OSM and EPO; OSM and REG1B; OSM and REG1A; OSM and CTSB; OSM and MMP12; OSM and CHI3L1; OSM and ULBP2; OSM and PRL; RNASE3 and FGF23; RNASE3 and CCL20; RNASE3 and EPO; RNASE3 and REG1B; RNASE3 and REG1A; RNASE3 and CTSB; RNASE3 and MMP12; RNASE3 and CHI3L1; RNASE3 and ULBP2; RNASE3 and PRL; TGFA and FGF23; TGFA and CCL20; TGFA and EPO; TGFA and REG1B; TGFA and REG1A; TGFA and CTSB; TGFA and MMP12; TGFA and CHI3L1; TGFA and ULBP2; TGFA and PRL; IL-6 and FGF23; IL-6 and CCL20; IL-6 and EPO; IL-6 and REG1B; IL-6 and REG1A; IL-6 and CTSB; IL-6 and MMP12; IL-6 and CHI3L1; IL-6 and ULBP2; IL-6 and PRL; AZU1 and FGF23; AZU1 and CCL20; AZU1 and EPO; AZU1 and REG1B; AZU1 and REG1A; AZU1 and CTSB; AZU1 and MMP12; AZU1 and CHI3L1; AZU1 and ULBP2; AZU1 and PRL; CD177 and FGF23; CD177 and CCL20; CD177 and EPO; CD177 and REG1B; CD177 and REG1A; CD177 and CTSB; CD177 and MMP12; CD177 and CHI3L1; CD177 and ULBP2; CD177 and PRL; CLEC4D and FGF23; CLEC4D and CCL20; CLEC4D and EPO; CLEC4D and REG1B; CLEC4D and REG1A; CLEC4D and CTSB; CLEC4D and MMP12; CLEC4D and CHI3L1; CLEC4D and ULBP2; CLEC4D and PRL.

Particular combinations of Group 3 proteins include ADAM15 and TRAIL and IP-10 and CRP; AGER and TRAIL and IP-10 and CRP; AGR2 and TRAIL and IP-10 and CRP; AREG and TRAIL and IP-10 and CRP; ASAH2 and TRAIL and IP-10 and CRP; CBLN4 and TRAIL and IP-10 and CRP; CCL17 and TRAIL and IP-10 and CRP; CCL24 and TRAIL and IP-10 and CRP; CCL8 and TRAIL and IP-10 and CRP; CD1C and TRAIL and IP-10 and CRP; CDH5 and TRAIL and IP-10 and CRP; CDON and TRAIL and IP-10 and CRP; CRTAC1 and TRAIL and IP-10 and CRP; CTSL and TRAIL and IP-10 and CRP; DDX58 and TRAIL and IP-10 and CRP; DSC2 and TRAIL and IP-10 and CRP; EZR and TRAIL and IP-10 and CRP; FBP1 and TRAIL and IP-10 and CRP; FCGR3B and TRAIL and IP-10 and CRP; GRPEL1 and TRAIL and IP-10 and CRP; IL-10 and TRAIL and IP-10 and CRP; KRT18 and TRAIL and IP-10 and CRP; MATN3 and TRAIL and IP-10 and CRP; MPHOSPH8 and TRAIL and IP-10 and CRP; NADK and TRAIL and IP-10 and CRP; P4HB and TRAIL and IP-10 and CRP; PLA2GA2 and TRAIL and IP-10 and CRP; POLR2F and TRAIL and IP-10 and CRP; PQBP1 and TRAIL and IP-10 and CRP; PTS and TRAIL and IP-10 and CRP; QPCT and TRAIL and IP-10 and CRP; REG1A and TRAIL and IP-10 and CRP; REG1B and TRAIL and IP-10 and CRP; RRM2 and TRAIL and IP-10 and CRP; SFTPA1 and TRAIL and IP-10 and CRP; SIGLEC6 and TRAIL and IP-10 and CRP; SIT1 and TRAIL and IP-10 and CRP; TNXB and TRAIL and IP-10 and CRP; TRIAP1 and TRAIL and IP-10 and CRP; TRIM21 and TRAIL and IP-10 and CRP; UMOD and TRAIL and IP-10 and CRP.

Particular Group 4 combinations include FGF23 and PLA2G2A; FGF23 and PTS; FGF23 and SFTPA1; FGF23 and EZR; FGF23 and SPP1; FGF23 and SCRN1; FGF23 and DDAH1; FGF23 and SFTPA2; FGF23 and POLR2F; IL-10 and PLA2G2A; IL-10 and PTS; IL-10 and SFTPA1; IL-10 and EZR; IL-10 and SPP1; IL-10 and SCRN1; IL-10 and DDAH1; IL-10 and SFTPA2; IL-10 and POLR2F; CCL20 and PLA2G2A; CCL20 and PTS; CCL20 and SFTPA1; CCL20 and EZR; CCL20 and SPP1; CCL20 and SCRN1; CCL20 and DDAH1; CCL20 and SFTPA2; CCL20 and POLR2F; CALCA and PLA2G2A; CALCA and PTS; CALCA and SFTPA1; CALCA and EZR; CALCA and PRDX1; CALCA and SCRN1; CALCA and DDAH1; CALCA and SFTPA2; CALCA and POLR2F; IL-6 and PLA2G2A; IL-6 and PTS; IL-6 and SFTPA1; IL-6 and EZR; IL-6 and PRDX1; IL-6 and SCRN1; IL-6 and DDAH1; IL-6 and SFTPA2; IL-6 and POLR2F; CXCL8 and PLA2G2A; CXCL8 and PTS; CXCL8 and SFTPA1; CXCL8 and EZR; CXCL8 and SPP1; CXCL8 and SCRN1; CXCL8 and DDAH1; CXCL8 and SFTPA2; CXCL8 and POLR2F; IL1RL1 and PLA2G2A; IL1RL1 and PTS; IL1RL1 and SFTPA1; IL1RL1 and EZR; IL1RL1 and PRDX1; IL1RL1 and SPP1; IL1RL1 and SCRN1; IL1RL1 and DDAH1; IL1RL1 and SFTPA2; IL1RL1 and POLR2F; IL1RN and PLA2G2A; IL1RN and PTS; IL1RN and SFTPA1; IL1RN and EZR; IL1RN and PRDX1; IL1RN and SPP1; IL1RN and SCRN1; IL1RN and DDAH1; IL1RN and SFTPA2; IL1RN and POLR2F; TNFRSF10B and PLA2G2A; TNFRSF10B and PTS; TNFRSF10B and SFTPA1; TNFRSF10B and EZR; TNFRSF10B and PRDX1; TNFRSF10B and SPP1; TNFRSF10B and SCRN1; TNFRSF10B and DDAH1; TNFRSF10B and SFTPA2; TNFRSF10B and POLR2F; STC1 and PLA2G2A; STC1 and PTS; STC1 and SFTPA1; STC1 and EZR; STC1 and SPP1; STC1 and SCRN1; STC1 and DDAH1; STC1 and SFTPA2; STC1 and POLR2F.

Additional combinations contemplated by the present inventors include FGF23 and KRT19; FGF23 and CCL7; FGF23 and FBP1; FGF23 and AGR2; FGF23 and RRM2; FGF23 and GRPEL1; FGF23 and TRIM21; FGF23 and DDX58; FGF23 and KRT18; FGF23 and AGER; IL-10 and KRT19; IL-10 and CCL7; IL-10 and FBP1; IL-10 and AGR2; IL-10 and RRM2; IL-10 and GRPEL1; IL-10 and TRIM21; IL-10 and DDX58; IL-10 and KRT18; IL-10 and AGER; CCL20 and KRT19; CCL20 and CCL7; CCL20 and FBP1; CCL20 and AGR2; CCL20 and RRM2; CCL20 and GRPEL1; CCL20 and TRIM21; CCL20 and DDX58; CCL20 and KRT18; CCL20 and AGER; CALCA and KRT19; CALCA and CCL7; CALCA and FBP1; CALCA and AGR2; CALCA and RRM2; CALCA and GRPEL1; CALCA and TRIM21; CALCA and DDX58; CALCA and KRT18; CALCA and AGER; IL-6 and KRT19; IL-6 and CCL7; IL-6 and FBP1; IL-6 and AGR2; IL-6 and RRM2; IL-6 and GRPEL1; IL-6 and TRIM21; IL-6 and DDX58; IL-6 and KRT18; IL-6 and AGER; CXCL8 and KRT19; CXCL8 and CCL7; CXCL8 and FBP1; CXCL8 and AGR2; CXCL8 and RRM2; CXCL8 and GRPEL1; CXCL8 and TRIM21; CXCL8 and DDX58; CXCL8 and KRT18; CXCL8 and AGER; IL1RL1 and KRT19; IL1RL1 and CCL7; IL1RL1 and FBP1; IL1RL1 and AGR2; IL1RL1 and RRM2; IL1RL1 and GRPEL1; IL1RL1 and TRIM21; IL1RL1 and DDX58; IL1RL1 and KRT18; IL1RL1 and AGER; IL1RN and KRT19; IL1RN and CCL7; IL1RN and FBP1; IL1RN and AGR2; IL1RN and RRM2; IL1RN and GRPEL1; IL1RN and TRIM21; IL1RN and DDX58; IL1RN and KRT18; IL1RN and AGER; TNFRSF10B and KRT19; TNFRSF10B and CCL7; TNFRSF10B and FBP1; TNFRSF10B and AGR2; TNFRSF10B and RRM2; TNFRSF10B and GRPEL1; TNFRSF10B and TRIM21; TNFRSF10B and DDX58; TNFRSF10B and KRT18; TNFRSF10B and AGER; STC1 and KRT19; STC1 and CCL7; STC1 and FBP1; STC1 and AGR2; STC1 and RRM2; STC1 and GRPEL1; STC1 and TRIM21; STC1 and DDX58; STC1 and KRT18; STC1 and AGER.

Further combinations contemplated by the present inventors include FGF23 and SIT1; FGF23 and CRTAC1; FGF23 and CDON; FGF23 and CCL17; FGF23 and TNFRSF10C; FGF23 and CD1C; FGF23 and DSC2; FGF23 and FCGR3B; FGF23 and QPCT; FGF23 and TNXB; IL-10 and SIT1; IL-10 and CRTAC1; IL-10 and CDON; IL-10 and CCL17; IL-10 and TNFRSF10C; IL-10 and CD1C; IL-10 and DSC2; IL-10 and FCGR3B; IL-10 and QPCT; IL-10 and TNXB; CCL20 and SIT1; CCL20 and CRTAC1; CCL20 and CDON; CCL20 and CCL17; CCL20 and TNFRSF10C; CCL20 and CD1C; CCL20 and DSC2; CCL20 and FCGR3B; CCL20 and QPCT; CCL20 and TNXB; CALCA and SIT1; CALCA and CRTAC1; CALCA and CDON; CALCA and CCL17; CALCA and CD1C; CALCA and DSC2; CALCA and FCGR3B; CALCA and QPCT; CALCA and TNXB; IL-6 and SIT1; IL-6 and CRTAC1; IL-6 and CDON; IL-6 and CCL17; IL-6 and CD1C; IL-6 and DSC2; IL-6 and FCGR3B; IL-6 and QPCT; IL-6 and TNXB; CXCL8 and SIT1; CXCL8 and CRTAC1; CXCL8 and CDON; CXCL8 and CCL17; CXCL8 and TNFRSF10C; CXCL8 and CD1C; CXCL8 and DSC2; CXCL8 and FCGR3B; CXCL8 and QPCT; CXCL8 and TNXB; IL1RL1 and SIT1; IL1RL1 and CRTAC1; IL1RL1 and CDON; IL1RL1 and CCL17; IL1RL1 and TNFRSF10C; IL1RL1 and CD1C; IL1RL1 and DSC2; IL1RL1 and FCGR3B; IL1RL1 and QPCT; IL1RL1 and TNXB; IL1RN and SIT1; IL1RN and CRTAC1; IL1RN and CDON; IL1RN and CCL17; IL1RN and CD1C; IL1RN and DSC2; IL1RN and FCGR3B; IL1RN and QPCT; IL1RN and TNXB; TNFRSF10B and SIT1; TNFRSF10B and CRTAC1; TNFRSF10B and CDON; TNFRSF10B and CCL17; TNFRSF10B and TNFRSF10C; TNFRSF10B and CD1C; TNFRSF10B and DSC2; TNFRSF10B and FCGR3B; TNFRSF10B and QPCT; TNFRSF10B and TNXB; STC1 and SIT1; STC1 and CRTAC1; STC1 and CDON; STC1 and CCL17; STC1 and TNFRSF10C; STC1 and CD1C; STC1 and DSC2; STC1 and FCGR3B; STC1 and QPCT; STC1 and TNXB.

Particular Group 5 protein combinations include IL-6 and PM20D1; IL-6 and IFNG; IL-6 and IL-10; IL-6 and DDX58; IL-6 and CXCL11; IL-6 and SIGLEC5; IL-6 and NADK; IL-6 and CCL8; IL-6 and PPP1R9B; IL-6 and SIGLEC1; PLA2G2A and PM20D1; PLA2G2A and IFNG; PLA2G2A and IL-10; PLA2G2A and DDX58; PLA2G2A and CXCL11; PLA2G2A and SIGLEC5; PLA2G2A and NADK; PLA2G2A and CCL8; PLA2G2A and PPP1R9B; PLA2G2A and SIGLEC1; CSF3 and PM20D1; CSF3 and IFNG; CSF3 and IL-10; CSF3 and DDX58; CSF3 and CXCL11; CSF3 and SIGLEC5; CSF3 and NADK; CSF3 and CCL8; CSF3 and PPP1R9B; CSF3 and SIGLEC1; PRTN3 and PM20D1; PRTN3 and IFNG; PRTN3 and IL-10; PRTN3 and DDX58; PRTN3 and CXCL11; PRTN3 and SIGLEC5; PRTN3 and NADK; PRTN3 and CCL8; PRTN3 and PPP1R9B; PRTN3 and SIGLEC1; MMP8 and PM20D1; MMP8 and IFNG; MMP8 and IL-10; MMP8 and DDX58; MMP8 and CXCL11; MMP8 and SIGLEC5; MMP8 and NADK; MMP8 and CCL8; MMP8 and PPP1R9B; MMP8 and SIGLEC1; LBP and PM20D1; LBP and IFNG; LBP and IL-10; LBP and DDX58; LBP and CXCL11; LBP and SIGLEC5; LBP and NADK; LBP and CCL8; LBP and PPP1R9B; LBP and SIGLEC1; VWA1 and PM20D1; VWA1 and IFNG; VWA1 and IL-10; VWA1 and DDX58; VWA1 and CXCL11; VWA1 and SIGLEC5; VWA1 and NADK; VWA1 and CCL8; VWA1 and PPP1R9B; VWA1 and SIGLEC1; OSM and PM20D1; OSM and IFNG; OSM and IL-10; OSM and DDX58; OSM and CXCL11; OSM and SIGLEC5; OSM and NADK; OSM and CCL8; OSM and PPP1R9B; OSM and SIGLEC1; GPR37 and PM20D1; GPR37 and IFNG; GPR37 and IL-10; GPR37 and DDX58; GPR37 and CXCL11; GPR37 and SIGLEC5; GPR37 and NADK; GPR37 and CCL8; GPR37 and PPP1R9B; GPR37 and SIGLEC1; IL1RN and PM20D1; IL1RN and IFNG; IL1RN and IL-10; IL1RN and DDX58; IL1RN and SIGLEC5; IL1RN and NADK; IL1RN and CCL8; IL1RN and PPP1R9B; IL1RN and SIGLEC1.

Particular Group 6 protein combinations include PM20D1 and IP-10 and CRP; IL-6 and IP-10 and CRP; PLA2G2A and IP-10 and CRP; IFNG and IP-10 and CRP; PRTN3 and IP-10 and CRP; CXCL10 (IP-10) and IP-10 and CRP; LBP and IP-10 and CRP; VWA1 and IP-10 and CRP; OSM and IP-10 and CRP; IL-10 and IP-10 and CRP; GPR37 and IP-10 and CRP; AGXT and IP-10 and CRP; C4BPB and IP-10 and CRP; AZU1 and IP-10 and CRP; DEFA1_DEFA1B and IP-10 and CRP; SERPINB8 and IP-10 and CRP; RRM2 and IP-10 and CRP; NADK and IP-10 and CRP; RNASE3 and IP-10 and CRP; PIK3AP1 and IP-10 and CRP; HCLS1 and IP-10 and CRP; LCN2 and IP-10 and CRP; SLAMF7 and IP-10 and CRP; CD14 and IP-10 and CRP; SHMT1 and IP-10 and CRP; SERPINB1 and IP-10 and CRP; CLEC6A and IP-10 and CRP; IL1B and IP-10 and CRP; CLEC4D and IP-10 and CRP; AHCY and IP-10 and CRP; CEACAM8 and IP-10 and CRP; LIF and IP-10 and CRP; FKBP5 and IP-10 and CRP; EGLN1 and IP-10 and CRP; CASP10 and IP-10 and CRP; B4GALT1 and IP-10 and CRP; CCL23 and IP-10 and CRP; PXN and IP-10 and CRP; IPCEF1 and IP-10 and CRP; IL10RA and IP-10 and CRP; STC1 and IP-10 and CRP; GZMB and IP-10 and CRP; TYMP and IP-10 and CRP; TXLNA and IP-10 and CRP; IL15 and IP-10 and CRP; LRIG1 and IP-10 and CRP; CXCL13 and IP-10 and CRP; RETN and IP-10 and CRP; SIRPB1 and IP-10 and CRP; SAMD9L and IP-10 and CRP; FYB1 and IP-10 and CRP; CD300E and IP-10 and CRP; SELE and IP-10 and CRP; FCN2 and IP-10 and CRP; CCL7 and IP-10 and CRP; LILRA5 and IP-10 and CRP; CXCL3 and IP-10 and CRP; TNFRSF8 and IP-10 and CRP; CSF1 and IP-10 and CRP; NOS3 and IP-10 and CRP; MPO and IP-10 and CRP; ICAM2 and IP-10 and CRP; ST6GAL1 and IP-10 and CRP; PAG1 and IP-10 and CRP; MCFD2 and IP-10 and CRP; BCL2L11 and IP-10 and CRP; SLC39A14 and IP-10 and CRP; PGLYRP1 and IP-10 and CRP; SORD and IP-10 and CRP; FCAR and IP-10 and CRP; EFNA1 and IP-10 and CRP; PTPN6 and IP-10 and CRP; MILR1 and IP-10 and CRP; SNAP29 and IP-10 and CRP; CCL18 and IP-10 and CRP; GNLY and IP-10 and CRP; USP8 and IP-10 and CRP; SKAP2 and IP-10 and CRP; NUDC and IP-10 and CRP; FLT4 and IP-10 and CRP; IKBKG and IP-10 and CRP; ICAM1 and IP-10 and CRP; BACH1 and IP-10 and CRP; CLEC4G and IP-10 and CRP; SEMA3F and IP-10 and CRP; LAT2 and IP-10 and CRP; TPP1 and IP-10 and CRP; CD300LF and IP-10 and CRP; TNFRSF1A and IP-10 and CRP; TNF and IP-10 and CRP; TARBP2 and IP-10 and CRP; IL2RA and IP-10 and CRP; TIMD4 and IP-10 and CRP; DDX58 and IP-10 and CRP; NBN and IP-10 and CRP; TNFSF13B and IP-10 and CRP; RARRES2 and IP-10 and CRP; PTPN1 and IP-10 and CRP; GBP4 and IP-10 and CRP; ANGPTL2 and IP-10 and CRP; GOLM2 and IP-10 and CRP; GRN and IP-10 and CRP; SIGLEC1 and IP-10 and CRP; PTK7 and IP-10 and CRP; C1QA and IP-10 and CRP; IL18BP and IP-10 and CRP; FOLR2 and IP-10 and CRP; GGH and IP-10 and CRP; SOD2 and IP-10 and CRP; LILRB1 and IP-10 and CRP; LYN and IP-10 and CRP; TXNDC15 and IP-10 and CRP; DECR1 and IP-10 and CRP; F9 and IP-10 and CRP; TIE1 and IP-10 and CRP; YES1 and IP-10 and CRP; C2 and IP-10 and CRP; FCGR3B and IP-10 and CRP; IMPA1 and IP-10 and CRP; SEMA4D and IP-10 and CRP; ADAM8 and IP-10 and CRP; SIGLEC9 and IP-10 and CRP; CA4 and IP-10 and CRP; VCAN and IP-10 and CRP; PLAU and IP-10 and CRP; IL13RA1 and IP-10 and CRP; TIA1 and IP-10 and CRP; ROBO2 and IP-10 and CRP; HYAL1 and IP-10 and CRP; BOC and IP-10 and CRP; MCAM and IP-10 and CRP; PRTG and IP-10 and CRP; GUCA2A and IP-10 and CRP; DDC and IP-10 and CRP; CDON and IP-10 and CRP; IL22RA1 and IP-10 and CRP; BMP4 and IP-10 and CRP; CES3 and IP-10 and CRP; HSD11B1 and IP-10 and CRP; GDF2 and IP-10 and CRP; DCBLD2 and IP-10 and CRP; EPCAM and IP-10 and CRP; CCL25 and IP-10 and CRP; CCN1 and IP-10 and CRP; CPM and IP-10 and CRP; ISM1 and IP-10 and CRP; NPTX1 and IP-10 and CRP; SERPINA12 and IP-10 and CRP; LGALS4 and IP-10 and CRP; TCL1A and IP-10 and CRP; EPHA1 and IP-10 and CRP; CTSV and IP-10 and CRP; CRH and IP-10 and CRP; CTSF and IP-10 and CRP; TNFSF11 and IP-10 and CRP; SIGLEC5 and IP-10 and CRP; CCL8 and IP-10 and CRP; PPP1R9B and IP-10 and CRP; TRIM21 and IP-10 and CRP; ITM2A and IP-10 and CRP; BANK1 and IP-10 and CRP; LAMP3 and IP-10 and CRP; NUB1 and IP-10 and CRP; BCR and IP-10 and CRP; GZMH and IP-10 and CRP; FEN1 and IP-10 and CRP; APBB1IP and IP-10 and CRP; CNST and IP-10 and CRP; IL12B and IP-10 and CRP; LAG3 and IP-10 and CRP; PPP1R12A and IP-10 and CRP; LAP3 and IP-10 and CRP; AIF1 and IP-10 and CRP; ARHGAP25 and IP-10 and CRP; INPPL1 and IP-10 and CRP; TDRKH and IP-10 and CRP; MMP13 and IP-10 and CRP; LGALS9 and IP-10 and CRP; PRKAR1A and IP-10 and CRP; AXIN1 and IP-10 and CRP; CASP3 and IP-10 and CRP; CERT and IP-10 and CRP; CPPED1 and IP-10 and CRP; RHOC and IP-10 and CRP; PPP1R2 and IP-10 and CRP; COMT and IP-10 and CRP; KIFBP and IP-10 and CRP; IL17RA and IP-10 and CRP; CLUL1 and IP-10 and CRP; S100A11 and IP-10 and CRP; FOXO1 and IP-10 and CRP; ILKAP and IP-10 and CRP; BST2 and IP-10 and CRP; TIGAR and IP-10 and CRP; NFKBIE and IP-10 and CRP; ADA2 and IP-10 and CRP; GRAP2 and IP-10 and CRP; TBL1X and IP-10 and CRP; FASLG and IP-10 and CRP; AXL and IP-10 and CRP; MANSCI and IP-10 and CRP; DPP4 and IP-10 and CRP; CD34 and IP-10 and CRP; ENTPD5 and IP-10 and CRP; CD244 and IP-10 and CRP; SLITRK6 and IP-10 and CRP; TSPAN1 and IP-10 and CRP; VNN2 and IP-10 and CRP; CCL16 and IP-10 and CRP; MMP9 and IP-10 and CRP; ASAH2 and IP-10 and CRP; HBEGF and IP-10 and CRP; KLK12 and IP-10 and CRP.

In order to diagnose infections (e.g. determine severity and/or distinguish between bacterial and viral infection, using more than one protein determinant, the threshold levels provided herein above may be used. Alternatively, scores based on the amounts of these proteins may be generated which take into account the weights of each of the proteins, as further described herein below.

Preferably the combinations which are tested to classify the infectious disease do not exceed 50, 40, 30, 20, 10, 9, 8, 7, 6, 5, 4, 3, or 2 markers. In another embodiment, no more than 40 protein markers are analyzed in a single test/analysis for the classification. In another embodiment, no more than 30 protein markers are analyzed in a single test/analysis for the classification. In another embodiment, no more than 20 protein markers are analyzed in a single test/analysis for the classification. In another embodiment, no more than 10 protein markers are analyzed in a single test/analysis for the classification. In another embodiment, no more than 9 protein markers are analyzed in a single test/analysis for the classification. In another embodiment, no more than 8 protein markers are analyzed in a single test/analysis for the classification. In another embodiment, no more than 7 protein markers are analyzed in a single test/analysis for the classification. In another embodiment, no more than 6 protein markers are analyzed in a single test/analysis, for the classification. In another embodiment, no more than 5 protein markers are analyzed in a single test/analysis for the classification. In another embodiment, no more than 4 protein markers are analyzed in a single test/analysis for the classification. In another embodiment, no more than 3 protein markers are analyzed in a single test/analysis for the classification. In another embodiment, no more than 2 protein markers are analyzed in a single test/analysis for the classification.

### Performance and Accuracy Measures of the Invention

The performance and thus absolute and relative clinical usefulness of the invention may be assessed in multiple ways as noted above. Amongst the various assessments of performance, some aspects of the invention are intended to provide accuracy in clinical diagnosis and prognosis. The accuracy of a diagnostic or prognostic test, assay, or method concerns the ability of the test, assay, or method to distinguish between subjects having an infection is based on whether the subjects have, a “significant alteration” (e.g., clinically significant and diagnostically significant) in the levels of a determinant. By “effective amount” it is meant that the measurement of an appropriate number of determinants (which may be one or more) to produce a “significant alteration” (e.g. level of expression or activity of a determinant) that is different than the predetermined cut-off point (or threshold value) for that determinant (s) and therefore indicates that the subject has an infection for which the determinant (s) is an indication. The difference in the level of determinant is preferably statistically significant. As noted below, and without any limitation of the invention, achieving statistical significance, and thus the preferred analytical, diagnostic, and clinical accuracy, may require that combinations of several determinants be used together in panels and combined with mathematical algorithms in order to achieve a statistically significant determinant index.

In the categorical diagnosis of a disease state, changing the cut point or threshold value of a test (or assay) usually changes the sensitivity and specificity, but in a qualitatively inverse relationship. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points. One way to achieve this is by using the Matthews correlation coefficient (MCC) metric, which depends upon both sensitivity and specificity. Use of statistics such as area under the ROC curve (AUC), encompassing all potential cut point values, is preferred for most categorical risk measures when using some aspects of the invention, while for continuous risk measures, statistics of goodness-of-fit and calibration to observed results or other gold standards, are preferred.

By predetermined level of predictability it is meant that the method provides an acceptable level of clinical or diagnostic accuracy. Using such statistics, an “acceptable degree of diagnostic accuracy”, is herein defined as a test or assay (such as the test used in some aspects of the invention for determining the clinically significant presence of determinants, which thereby indicates the presence of an infection type and/or the severity of the infection) in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.75, 0.80, desirably at least 0.85, more desirably at least 0.875, preferably at least 0.90, more preferably at least 0.925, and most preferably at least 0.95.

Alternatively, the methods predict the presence or absence of an infection or severity of infection with at least 75% total accuracy, more preferably 80%, 85%, 90%, 95%, 97%, 98%, 99% or greater total accuracy.

Alternatively, the methods predict the presence of a bacterial infection or response to therapy or severity of bacterial infection with at least 75% sensitivity, more preferably 80%, 85%, 90%, 95%, 97%, 98%, 99% or greater sensitivity.

Alternatively, the methods predict the presence of a viral infection or response to therapy or severity of viral infection with at least 75% specificity, more preferably 80%, 85%, 90%, 95%, 97%, 98%, 99% or greater specificity. Alternatively, the methods predict the presence or absence of an infection or response to therapy with an MCC larger than 0.2, 0.3, 0.4, 0.5, 0.6, 0.7, 0.8, 0.9 or 1.0.

In general, alternative methods of determining diagnostic accuracy are commonly used for continuous measures, when a disease category has not yet been clearly defined by the relevant medical societies and practice of medicine, where thresholds for therapeutic use are not yet established, or where there is no existing gold standard for diagnosis of the pre-disease. For continuous measures of risk, measures of diagnostic accuracy for a calculated index are typically based on curve fit and calibration between the predicted continuous value and the actual observed values (or a historical index calculated value) and utilize measures such as R squared, Hosmer-Lemeshow P-value statistics and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health, Inc. (Redwood City, California).

In general, by defining the degree of diagnostic accuracy, i.e., cut points on a ROC curve, defining an acceptable AUC value, and determining the acceptable ranges in relative concentration of what constitutes an effective amount of the determinants of the invention allows for one of skill in the art to use the determinants to identify, diagnose, or prognose subjects with a pre-determined level of predictability and performance.

Furthermore, other unlisted biomarkers will be very highly correlated with the determinants (for the purpose of this application, any two variables will be considered to be “very highly correlated” when they have a Coefficient of Determination (R²) of 0.5 or greater). Some aspects of the present invention encompass such functional and statistical equivalents to the aforementioned determinants. Furthermore, the statistical utility of such additional determinants is substantially dependent on the cross-correlation between multiple biomarkers and any new biomarkers will often be required to operate within a panel in order to elaborate the meaning of the underlying biology.

### Construction of Determinant Panels

Groupings of determinants can be included in “panels”, also called “determinant-signatures”, “determinant signatures”, or “multi-determinant signatures.” A “panel” within the context of the present invention means a group of biomarkers (whether they are determinants, clinical parameters, or traditional laboratory risk factors) that includes one or more determinants. A panel can also comprise additional biomarkers, e.g., clinical parameters, traditional laboratory risk factors, known to be present or associated with infection, in combination with a selected group of the determinants listed herein.

As noted above, many of the individual determinants, clinical parameters, and traditional laboratory risk factors listed, when used alone and not as a member of a multi-biomarker panel of determinants, have little or no clinical use in reliably distinguishing individual normal subjects, subjects at risk for having an infection (e.g., bacterial, viral or co-infection), or severity of infection and thus cannot reliably be used alone in classifying any subject between those states. Even where there are statistically significant differences in their mean measurements in each of these populations, as commonly occurs in studies which are sufficiently powered, such biomarkers may remain limited in their applicability to an individual subject, and contribute little to diagnostic or prognostic predictions for that subject. A common measure of statistical significance is the p-value, which indicates the probability that an observation has arisen by chance alone; preferably, such p-values are 0.05 or less, representing a 5% or less chance that the observation of interest arose by chance. Such p-values depend significantly on the power of the study performed.

Despite this individual determinant performance, and the general performance of formulas combining only the traditional clinical parameters and few traditional laboratory risk factors, the present inventors have noted that certain specific combinations of two or more determinants can also be used as multi-biomarker panels comprising combinations of determinants that are known to be involved in one or more physiological or biological pathways, and that such information can be combined and made clinically useful through the use of various formulae, including statistical classification algorithms and others, combining and in many cases extending the performance characteristics of the combination beyond that of the individual determinants. These specific combinations show an acceptable level of diagnostic accuracy, and, when sufficient information from multiple determinants is combined in a trained formula, they often reliably achieve a high level of diagnostic accuracy transportable from one population to another.

The general concept of how two less specific or lower performing determinants are combined into novel and more useful combinations for the intended indications, is a key aspect of some embodiments of the invention. Multiple biomarkers can yield significant improvement in performance compared to the individual components when proper mathematical and clinical algorithms are used; this is often evident in both sensitivity and specificity, and results in a greater AUC or MCC. Significant improvement in performance could mean an increase of 1%, 2%, 3%, 4%, 5%, 8%, 10% or higher than 10% in different measures of accuracy such as total accuracy, AUC, MCC, sensitivity, specificity, PPV or NPV. Secondly, there is often novel unperceived information in the existing biomarkers, as such was necessary in order to achieve through the new formula an improved level of sensitivity or specificity. This hidden information may hold true even for biomarkers which are generally regarded to have suboptimal clinical performance on their own. In fact, the suboptimal performance in terms of high false positive rates on a single biomarker measured alone may very well be an indicator that some important additional information is contained within the biomarker results—information which would not be elucidated absent the combination with a second biomarker and a mathematical formula.

On the other hand, it is often useful to restrict the number of measured diagnostic determinants (e.g., protein markers), as this allows significant cost reduction and reduces required sample volume and assay complexity. Accordingly, even when two signatures have similar diagnostic performance (e.g., similar AUC or sensitivity), one which incorporates less proteins could have significant utility and ability to reduce to practice. For example, a signature that includes 5 proteins compared to 10 proteins and performs similarly has many advantages in real world clinical setting and thus is desirable. Therefore, there is value and invention in being able to reduce the number of proteins incorporated in a signature while retaining similar levels of accuracy. In this context similar levels of accuracy could mean plus or minus 1%, 2%, 3%, 4%, 5%, 8%, or 10% in different measures of accuracy such as total accuracy, AUC, MCC, sensitivity, specificity, PPV or NPV; a significant reduction in the number of proteins of a signature includes reducing the number of proteins by 2, 3, 4, 5, 6, 7, 8, 9, 10 or more than 10 proteins.

Several statistical and modeling algorithms known in the art can be used to both assist in determinant selection choices and optimize the algorithms combining these choices. Statistical tools such as factor and cross-biomarker correlation/covariance analyses allow more rationale approaches to panel construction. Mathematical clustering and classification tree showing the Euclidean standardized distance between the determinants can be advantageously used. Pathway informed seeding of such statistical classification techniques also may be employed, as may rational approaches based on the selection of individual determinants based on their participation across in particular pathways or physiological functions.

Ultimately, formula such as statistical classification algorithms can be directly used to both select determinants and to generate and train the optimal formula necessary to combine the results from multiple determinants into a single index. Often, techniques such as forward (from zero potential explanatory parameters) and backwards selection (from all available potential explanatory parameters) are used, and information criteria, such as AIC or BIC, are used to quantify the tradeoff between the performance and diagnostic accuracy of the panel and the number of determinants used. The position of the individual determinant on a forward or backwards selected panel can be closely related to its provision of incremental information content for the algorithm, so the order of contribution is highly dependent on the other constituent determinants in the panel.

### Construction of Clinical Algorithms

Any formula may be used to combine determinant results into indices useful in the practice of the invention. As indicated above, and without limitation, such indices may indicate, among the various other indications, the probability, likelihood, absolute or relative risk, time to or rate of conversion from one to another disease states, or make predictions of future biomarker measurements of infection. This may be for a specific time period or horizon, or for remaining lifetime risk, or simply be provided as an index relative to another reference subject population.

Although various preferred formula are described here, several other model and formula types beyond those mentioned herein and in the definitions above are well known to one skilled in the art. The actual model type or formula used may itself be selected from the field of potential models based on the performance and diagnostic accuracy characteristics of its results in a training population. The specifics of the formula itself may commonly be derived from determinant results in the relevant training population. Amongst other uses, such formula may be intended to map the feature space derived from one or more determinant inputs to a set of subject classes (e.g. useful in predicting class membership of subjects as normal, having an infection), to derive an estimation of a probability function of risk using a Bayesian approach, or to estimate the class-conditional probabilities, then use Bayes' rule to produce the class probability function as in the previous case.

Preferred formulas include the broad class of statistical classification algorithms, and in particular the use of discriminant analysis. The goal of discriminant analysis is to predict class membership from a previously identified set of features. In the case of linear discriminant analysis (LDA), the linear combination of features is identified that maximizes the separation among groups by some criteria. Features can be identified for LDA using an eigengene based approach with different thresholds (ELDA) or a stepping algorithm based on a multivariate analysis of variance (MANOVA). Forward, backward, and stepwise algorithms can be performed that minimize the probability of no separation based on the Hotelling-Lawley statistic.

Eigengene-based Linear Discriminant Analysis (ELDA) is a feature selection technique developed by Shen et al. (2006). The formula selects features (e.g. biomarkers) in a multivariate framework using a modified eigen analysis to identify features associated with the most important eigenvectors. “Important” is defined as those eigenvectors that explain the most variance in the differences among samples that are trying to be classified relative to some threshold.

A support vector machine (SVM) is a classification formula that attempts to find a hyperplane that separates two classes. This hyperplane contains support vectors, data points that are exactly the margin distance away from the hyperplane. In the likely event that no separating hyperplane exists in the current dimensions of the data, the dimensionality is expanded greatly by projecting the data into larger dimensions by taking non-linear functions of the original variables (Venables and Ripley, 2002). Although not required, filtering of features for SVM often improves prediction. Features (e.g., biomarkers) can be identified for a support vector machine using a non-parametric Kruskal-Wallis (KW) test to select the best univariate features. A random forest (RF, Breiman, 2001) or recursive partitioning (RPART, Breiman et al., 1984) can also be used separately or in combination to identify biomarker combinations that are most important. Both KW and RF require that a number of features be selected from the total. RPART creates a single classification tree using a subset of available biomarkers.

Other formula may be used in order to pre-process the results of individual determinant measurements into more valuable forms of information, prior to their presentation to the predictive formula. Most notably, normalization of biomarker results, using either common mathematical transformations such as logarithmic or logistic functions, as normal or other distribution positions, in reference to a population's mean values, etc. are all well known to those skilled in the art. Of particular interest are a set of normalizations based on clinical-determinants such as time from symptoms, gender, race, or sex, where specific formula are used solely on subjects within a class or continuously combining a clinical-determinants as an input. In other cases, analyte-based biomarkers can be combined into calculated variables which are subsequently presented to a formula.

In addition to the individual parameter values of one subject potentially being normalized, an overall predictive formula for all subjects, or any known class of subjects, may itself be recalibrated or otherwise adjusted based on adjustment for a population's expected prevalence and mean biomarker parameter values, according to the technique outlined in D'Agostino et al., (2001) JAMA 286:180-187, or other similar normalization and recalibration techniques. Such epidemiological adjustment statistics may be captured, confirmed, improved and updated continuously through a registry of past data presented to the model, which may be machine readable or otherwise, or occasionally through the retrospective query of stored samples or reference to historical studies of such parameters and statistics. Additional examples that may be the subject of formula recalibration or other adjustments include statistics used in studies by Pepe, M. S. et al., 2004 on the limitations of odds ratios; Cook, N. R., 2007 relating to ROC curves. Finally, the numeric result of a classifier formula itself may be transformed post-processing by its reference to an actual clinical population and study results and observed endpoints, in order to calibrate to absolute risk and provide confidence intervals for varying numeric results of the classifier or risk formula.

Some determinants may exhibit trends that depends on the patient age (e.g. the population baseline may rise or fall as a function of age). One can use a ‘Age dependent normalization or stratification’ scheme to adjust for age related differences. Performing age dependent normalization, stratification or distinct mathematical formulas can be used to improve the accuracy of determinants for differentiating between different types of infections. For example, one skilled in the art can generate a function that fits the population mean levels of each determinant as function of age and use it to normalize the determinant of individual subjects levels across different ages. Another example is to stratify subjects according to their age and determine age specific thresholds or index values for each age group independently.

In the context of the present invention the following statistical terms may be used:

“TP” is true positive, means positive test result that accurately reflects the tested-for activity. For example in the context of the present invention a TP, is for example but not limited to, truly classifying a bacterial infection as such.

“TN” is true negative, means negative test result that accurately reflects the tested-for activity. For example in the context of the present invention a TN, is for example but not limited to, truly classifying a viral infection as such.

“FN” is false negative, means a result that appears negative but fails to reveal a situation. For example in the context of the present invention a FN, is for example but not limited to, falsely classifying a bacterial infection as a viral infection.

“FP” is false positive, means test result that is erroneously classified in a positive category. For example in the context of the present invention a FP, is for example but not limited to, falsely classifying a viral infection as a bacterial infection.

“Sensitivity” is calculated by TP/(TP+FN) or the true positive fraction of disease subjects.

“Specificity” is calculated by TN/(TN+FP) or the true negative fraction of non-disease or normal subjects.
“Total accuracy” is calculated by (TN+TP)/(TN+FP+TP+FN).
“Positive predictive value” or “PPV” is calculated by TP/(TP+FP) or the true positive fraction of all positive test results. It is inherently impacted by the prevalence of the disease and pre-test probability of the population intended to be tested.
“Negative predictive value” or “NPV” is calculated by TN/(TN+FN) or the true negative fraction of all negative test results. It also is inherently impacted by the prevalence of the disease and pre-test probability of the population intended to be tested. See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, e.g., a clinical diagnostic test.

“MCC” (Mathews Correlation coefficient) is calculated as follows: MCC=(TP*TN−FP*FN)/{(TP+FN)*(TP+FP)*(TN+FP)*(TN+FN)}\hat{ }0.5 where TP, FP, TN, FN are true-positives, false-positives, true-negatives, and false-negatives, respectively. Note that MCC values range between −1 to +1, indicating completely wrong and perfect classification, respectively. An MCC of 0 indicates random classification. MCC has been shown to be a useful for combining sensitivity and specificity into a single metric (Baldi, Brunak et al. 2000). It is also useful for measuring and optimizing classification accuracy in cases of unbalanced class sizes (Baldi, Brunak et al. 2000).

Often, for binary disease state classification approaches using a continuous diagnostic test measurement, the sensitivity and specificity is summarized by a Receiver Operating Characteristics (ROC) curve according to Pepe et al., “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation Of Laboratory Procedures,” chapter 14 in Teitz, Fundamentals of Clinical Chemistry, Burtis and Ashwood (eds.), 4^{th} edition 1996, W.B. Saunders Company, pages 192-199; and Zweig et al., “ROC Curve Analysis: An Example Showing The Relationships Among Serum Lipid And Apolipoprotein Concentrations In Identifying Subjects With Coronary Artery Disease,” Clin. Chem., 1992, 38(8): 1425-1428. An alternative approach using likelihood functions, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), Mathews correlation coefficient (MCC), or as a likelihood, odds ratio, Receiver Operating Characteristic (ROC) curve, Area Under the Curve (AUC) among other measures.

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value”. Non-limiting examples of “formulas” include sums, ratios, and regression operators, such as coefficients or exponents, biomarker value transformations and normalizations (including, without limitation, those normalization schemes based on clinical-determinants, such as gender, age, or ethnicity), rules and guidelines, statistical classification models, and neural networks trained on historical populations. Of particular use in combining determinants are linear and non-linear equations and statistical classification analyses to determine the relationship between levels of determinants detected in a subject sample and the subject's probability of having an infection or a certain type of infection. In panel and combination construction, of particular interest are structural and syntactic statistical classification algorithms, and methods of index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, and Hidden Markov Models, among others. Other techniques may be used in survival and time to event hazard analysis, including Cox, Weibull, Kaplan-Meier and Greenwood models well known to those of skill in the art. Many of these techniques are useful either combined with a determinant selection technique, such as forward selection, backwards selection, or stepwise selection, complete enumeration of all potential panels of a given size, genetic algorithms, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art. A “health economic utility function” is a formula that is derived from a combination of the expected probability of a range of clinical outcomes in an idealized applicable patient population, both before and after the introduction of a diagnostic or therapeutic intervention into the standard of care. It encompasses estimates of the accuracy, effectiveness and performance characteristics of such intervention, and a cost and/or value measurement (a utility) associated with each outcome, which may be derived from actual health system costs of care (services, supplies, devices and drugs, etc.) and/or as an estimated acceptable value per quality adjusted life year (QALY) resulting in each outcome. The sum, across all predicted outcomes, of the product of the predicted population size for an outcome multiplied by the respective outcome's expected utility is the total health economic utility of a given standard of care. The difference between (i) the total health economic utility calculated for the standard of care with the intervention versus (ii) the total health economic utility for the standard of care without the intervention results in an overall measure of the health economic cost or value of the intervention. This may itself be divided amongst the entire patient group being analyzed (or solely amongst the intervention group) to arrive at a cost per unit intervention, and to guide such decisions as market positioning, pricing, and assumptions of health system acceptance.

Such health economic utility functions are commonly used to compare the cost-effectiveness of the intervention, but may also be transformed to estimate the acceptable value per QALY the health care system is willing to pay, or the acceptable cost-effective clinical performance characteristics required of a new intervention.

For diagnostic (or prognostic) interventions of the invention, as each outcome (which in a disease classifying diagnostic test may be a TP, FP, TN, or FN) bears a different cost, a health economic utility function may preferentially favor sensitivity over specificity, or PPV over NPV based on the clinical situation and individual outcome costs and value, and thus provides another measure of health economic performance and value which may be different from more direct clinical or analytical performance measures. These different measurements and relative trade-offs generally will converge only in the case of a perfect test, with zero error rate (a.k.a., zero predicted subject outcome misclassifications or FP and FN), which all performance measures will favor over imperfection, but to differing degrees.

“Analytical accuracy” refers to the reproducibility and predictability of the measurement process itself, and may be summarized in such measurements as coefficients of variation (CV), Pearson correlation, and tests of concordance and calibration of the same samples or controls with different times, users, equipment and/or reagents. These and other considerations in evaluating new biomarkers are also summarized in Vasan, 2006.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC and MCC, time to result, shelf life, etc. as relevant.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less.

### Kits

Some aspects of the invention also include a determinant-detection reagent such as antibodies packaged together in the form of a kit. The kit may contain in separate containers antibodies (either already bound to a solid matrix or packaged separately with reagents for binding them to the matrix), control formulations (positive and/or negative), and/or a detectable label such as fluorescein, green fluorescent protein, rhodamine, cyanine dyes, Alexa dyes, luciferase, radiolabels, among others. The detectable label may be attached to a secondary antibody which binds to the Fc portion of the antibody which recognizes the determinant. Instructions (e.g., written, tape, VCR, CD-ROM, etc.) for carrying out the assay may be included in the kit.

The kits of this aspect of the present invention may comprise additional components that aid in the detection of the determinants such as enzymes, salts, buffers etc. necessary to carry out the detection reactions.

For example, determinant detection reagents (e.g. antibodies) can be immobilized on a solid support such as a porous strip or an array to form at least one determinant detection site. The measurement or detection region of the porous strip may include a plurality of sites. A test strip may also contain sites for negative and/or positive controls. Alternatively, control sites can be located on a separate strip from the test strip. Optionally, the different detection sites may contain different amounts of immobilized detection reagents, e.g., a higher amount in the first detection site and lesser amounts in subsequent sites. Upon the addition of test sample, the number of sites displaying a detectable signal provides a quantitative indication of the amount of determinants present in the sample. The detection sites may be configured in any suitably detectable shape and are typically in the shape of a bar or dot spanning the width of a test strip.

Polyclonal antibodies for measuring determinants include without limitation antibodies that were produced from sera by active immunization of one or more of the following: Rabbit, Goat, Sheep, Chicken, Duck, Guinea Pig, Mouse, Donkey, Camel, Rat and Horse.

Examples of detection agents, include without limitation: scFv, dsFv, Fab, sVH, F(ab′)2, Cyclic peptides, Haptamers, A single-domain antibody, Fab fragments, Single-chain variable fragments, Affibody molecules, Affilins, Nanofitins, Anticalins, Avimers, DARPins, Kunitz domains, Fynomers and Monobody.

In particular embodiments, the kit does not comprise a number of antibodies that specifically recognize more than 50, 20 15, 10, 9, 8, 7, 6, 5 or 4 polypeptides.

In other embodiments, the array of the present invention does not comprise a number of antibodies that specifically recognize more than 50, 20 15, 10, 9, 8, 7, 6, 5 or 4 polypeptides.

In one embodiment, the kit comprises no more than 10, 9, 8, 7, 6, 6, 5, 4, 3 or 2 antibodies.

A machine-readable storage medium can comprise a data storage material encoded with machine-readable data or data arrays which, when using a machine programmed with instructions for using the data, is capable of use for a variety of purposes. Measurements of effective amounts of the biomarkers of the invention and/or the resulting evaluation of risk from those biomarkers can be implemented in computer programs executing on programmable computers, comprising, inter alia, a processor, a data storage system (including volatile and non-volatile memory and/or storage elements), at least one input device, and at least one output device. Program code can be applied to input data to perform the functions described above and generate output information. The output information can be applied to one or more output devices, according to methods known in the art. The computer may be, for example, a personal computer, microcomputer, or workstation of conventional design.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein. The health-related data management system used in some aspects of the invention may also be considered to be implemented as a computer-readable storage medium, configured with a computer program, where the storage medium so configured causes a computer to operate in a specific and predefined manner to perform various functions described herein.

The polypeptide determinants of the present invention, in some embodiments thereof, can be used to generate a “reference determinant profile” of those subjects who do not have an infection. The determinants disclosed herein can also be used to generate a “subject determinant profile” taken from subjects who have an infection. The subject determinant profiles can be compared to a reference determinant profile to diagnose or identify subjects with an infection. The subject determinant profile of different infection types can be compared to diagnose or identify the type of infection. The reference and subject determinant profiles of the present invention, in some embodiments thereof, can be contained in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history. The machine-readable media can also contain information relating to other disease-risk algorithms and computed indices such as those described herein.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

The study cohort is presented in Table 4.

TABLE 4

| Group | Number in group |
|---|---|
| Healthy | 12 |
| Non infectious | 10 |
| infectious | 81 |
| Bacterial | 37 |
| Viral | 44 |
| Severe | 28 |
| Non severe | 63 |

Some patients were included in more than one group.

As viral infections are predominated today by COVID-19, in some of the groups above, COVID-19 patients are included in the viral patients.

Disease etiology was established by applying a rigorous expert panel adjudication process.

All patients had a blood sample taken during their hospitalization or emergency department visit course. Comparisons between the patient groups were done for identifying differential markers in blood serum.

In addition to this cohort, 2 publicly-available datasets were used for the comparison of severe COVID-19 to non-severe COVID-19.

Severity endpoints definitions: Using specific biomarkers and/or signatures the following severe endpoints may be predicted: SIRS without infection, sepsis, severe sepsis, septic shock, max NEWS score, max SOFA score, lowest SaO2/FiO2, PaO2/FiO2 ratios. Specifically, prediction of the following severe outcomes: Vasopressors requirement, Invasive mechanical ventilation (TMV) Intensive Care Unit (ICU)/Stepdown unit/Emergency department (ED) monitoring ED-Length Of Stay (LOS), Hospital LOS, ICU LOS, Renal replacement therapy, Mortality (24 hours mortality, 3 days mortality, 7 days mortality, 14 days mortality, 28 days mortality, in-hospital mortality.

### Materials and Methods

Protein screening was performed using Olink Proteomics' PEA technology (Olink®. Explore 1536)). In total, 1472 proteins from four different panels (Cardiometabolic, Inflammation, Neurology and Oncology) were measured. The resulting protein measurements enable relative quantification, where the results are expressed as normalized protein expression (NPX) arbitrary units on a log 2-scale.

### Measures of Biomarker Performance:

Performance measures for differentiating between two patient groups included:
1. The area under the receiver operating characteristic curve (AUC)
2. The difference between group medians (delta), in NPX units.

Since NPX units are on a log 2-scale, the ratio between group medians (also known as fold change) can be calculated from NPX delta by exponentiation: ratio=2delta In each comparison between two patient groups, markers with AUC>0.8 were included in a list of top-performing markers, and the list prioritized based on NPX delta. The public COVID dataset was used to expand the list of top performing biomarkers: markers with AUC<0.8 in the main cohort, but with AUC>0.75 in the public cohort, were added to the list.

### Results

The following proteins were found to be differentially expressed in bacterial vs. viral infections with a high AUC.

TABLE 5

| Gene | UniProt | | |
|---|---|---|---|
| Symbol | ID | Biomarker | Direction |
| PLA2G2A | P14555 | sPLA2-IIA (Group IIA phospholipase | High in |
| | | A2); NPS-PLA2 | Bacterial |
| CSF3 | P09919 | CSF3, G-CSF (Granulocyte colony- | High in |
| | | stimulating factor); | Bacterial |
| | | Pluripoietin/Filgrastim/Lenograstim | |
| MMP8 | P22894 | MMP 8; PMNL-CL | High in |
| | | | Bacterial |
| OSM | P13725 | Oncostatin-M | High in |
| | | | Bacterial |
| RNASE3 | P12724 | Eosinophil cationic protein; ECP; RNS3 | High in |
| | | Eosinophil cationic protein; ECP; RNS3 | Bacterial |
| TGFA | P01135 | Protransforming growth factor alpha; | High in |
| | | Transforming growth factor alpha (TGF- | Bacterial |
| | | alpha) | |
| IL6/ | P05231 | Interleukin-6; IL-6; B-cell stimulatory | High in |
| IFNB2 | | factor 2 (BSF-2); CTL differentiation | Bacterial |
| | | factor (CDF); Hybridoma growth factor | |
| | | Interferon beta-2 (IFN-beta-2) | |
| AZU1 | P20160 | HBP (Heparin-binding protein); | High in |
| | | Azurocidin/CAP37 | Bacterial |
| CD177 | Q8N6Q3 | Human neutrophil alloantigen 2a (HNA- | High in |
| | | 2a); NB1 glycoprotein/Polycythemia | Bacterial |
| | | rubra vera protein 1; NB1; PRV1 | |
| CLEC4D | Q8WXI8 | C-type lectin domain family 4 member | High in |
| | | D; C-type lectin superfamily member 8; | Bacterial |
| | | CLECSF8; MCL | |
| FGF23 | Q9GZV9 | FGF23 | High in |
| | | | Bacterial |
| CCL20 | P78556 | CCL20; Beta-chemokine exodus-1; MIP- | High in |
| | | 3-alpha; LARC | Bacterial |
| EPO | P01588 | Erythropoietin; Epoetin | High in |
| | | | Bacterial |
| REG1B | P48304 | PSP-2; Lithostathine-1-beta | High in |
| | | | Bacterial |
| REGIA | P05451 | PSP (pancreatic stone protein) | High in |
| | | | Bacterial |
| CTSB | P07858 | CTSB (Cathepsin B); APPS | High in |
| | | | Bacterial |
| MMP12 | P39900 | Macrophage metalloelastase; MME; | High in |
| | | Macrophage elastase (ME; hME); Matrix | Bacterial |
| | | metalloproteinase-12 (MMP-12) | |
| CHI3L1 | P36222 | CHI3L1 (Chitinase-3-like protein 1) | High in |
| | | (YKL-40); hCGP-39 | Bacterial |
| ULBP2 | Q9BZM5 | UL16-binding protein 2; ALCAN-alpha/ | High in |
| | | NKG2D ligand 2 (N2DL-2; NKG2DL2)/ | Bacterial |
| | | ULBP2; N2DL2; RAET1H | |
| PRL | P01236 | Prolactin | Low in |
| | | | Bacterial |
| TNFSF10/ | P50591 | Tumor necrosis factor ligand superfamily | Low in |
| TRAIL | | member 10; Apo-2 ligand (Apo-2L); | Bacterial |
| | | Protein TRAIL | |
| CRP | P02741 | C-reactive protein | High in |
| | | | Bacterial |
| CXCL10 | P02778 | C-X-C motif chemokine 10; IP-10 | Low in |
| | | | Bacterial |

The proteins which showed the largest delta (log 2 of the fold change) were REG11B (delta 2.760628), FGF23 (delta 2.352782) and CCL20 (delta 2.256179).

Proteins listed in Table 6 were found to be differentially expressed in a severe vs non-severe infection with a high AUC.

TABLE 6

| | Uniprot | | |
|---|---|---|---|
| Gene Symbol | ID | Biomarker | Direction |
| FGF23 | Q9GZV9 | FGF23 | High in |
| | | | Severe |
| IL-10 | P22301 | Interleukin-10; IL-10; Cytokine | High in |
| | | synthesis inhibitory factor (CSIF) | Severe |
| CCL20 | P78556 | CCL20; Beta-chemokine exodus-1; | High in |
| | | MIP-3-alpha; LARC | Severe |
| CALCA | P01258 | (Calcitonin) Procalcitonin; PCT; | High in |
| | | CCP; PDN-21 | Severe |
| IL6/IFNB2 | P05231 | Interleukin-6; IL-6; B-cell stimulatory | High in |
| | | factor 2 (BSF-2); CTL differentiation | Severe |
| | | factor (CDF); Hybridoma growth | |
| | | factor (CDF); Hybridoma growth | |
| | | factor | |
| | | Interferon beta-2 (IFN-beta-2) | |
| CXCL8 | P10145 | CXCL8; IL8; GCP-1; MDNCF; | High in |
| | | MONAP; NAP-1; Protein 3-10C | Severe |
| IL1RL1/ | Q01638 | sST2; Interleukin-1 receptor-like 1, | High in |
| DER4/ST2/ | | IL-1 R4, IL-33R; Protein ST2 | Severe |
| T1 | | | |
| IL1RN | P18510 | IL-1ra, Anakinra , Interleukin-1 | High in |
| | | receptor antagonist protein | Severe |
| TNFRSF10B | O14763 | TRAIL-R2 (TNFRSF10B); Death | High in |
| | | receptor 5 (DR5); CD262 | Severe |
| STC1 | P52823 | Stanniocalcin-1 | High in |
| | | | Severe |
| PLA2G2A | P14555 | sPLA2-IIA (Group IIA phospholipase | High in |
| | | A2); NPS-PLA2 | Severe |
| PTS | Q03393 | PTS (6-pyruvoyl tetrahydrobiopterin | High in |
| | | synthase) | Severe |
| SFTPA1 | Q8IWL2 | SFTPA1 (Pulmonary surfactant- | High in |
| | | associated protein A1); PSP-A/SP-A1; | Severe |
| | | Alveolar proteinosis protein/Collectin-4 | |
| EZR | P15311 | EZR (Ezrin); Cytovillin; Villin-2; p81 | High in |
| | | | Severe |
| PRDX1 | Q06830 | Peroxiredoxin-1; Natural killer cell- | High in |
| | | enhancing factor A (NKEF-A); | Severe |
| | | Proliferation-associated gene protein | |
| | | (PAG); | |
| | | Thioredoxin peroxidase 2; | |
| | | Thioredoxin-dependent peroxide | |
| | | reductase 2; | |
| | | Thioredoxin-dependent peroxiredoxin | |
| | | 1 | |
| SPP1 | P10451 | SPP1 (Osteopontin); Bone | High in |
| | | SPP1 (Osteopontin); Bone | Severe |
| | | sialoprotein 1; Nephropontin; | |
| | | Uropontin | |
| SCRN1 | Q12765 | Secernin-1 | High in |
| | | | Severe |
| DDAH1 | O94760 | N(G),N(G)-dimethylarginine | High in |
| | | dimethylaminohydrolase 1; DDAH-1; | Severe |
| | | Dimethylarginine | |
| | | dimethylaminohydrolase 1; | |
| | | Dimethylargininase-1 | |
| SFTPA2 | Q8IWL1 | Pulmonary surfactant-associated | High in |
| | | protein A2; PSP-A; PSPA; SP-A; SP- | Severe |
| | | A2; 35 kDa pulmonary surfactant- | |
| | | associated protein; | |
| | | Alveolar proteinosis protein; | |
| | | Collectin-5 | |
| POLR2F | P61218 | POLR2F (DNA-directed RNA | High in |
| | | polymerases I, II, and III subunit | Severe |
| | | RPABC2); RPB14.4; RPC15; RPB6 | |
| | | homolog | |
| KRT19 | P08727 | KRT19 (Keratin 19) | High in |
| | | | Severe |
| CCL7 | P80098 | MCP-3 (CCL7); NC28 | High in |
| | | | Severe |
| FBP1 | P09467 | FBP1 (Fructose-1,6-bisphosphatase | High in |
| | | 1); FBPase 1 | Severe |
| AGR2 | O95994 | AGR2 (Anterior gradient protein 2 | High in |
| | | homolog); hAG-2; HPC8 | Severe |
| RRM2 | P31350 | Ribonucleoside-diphosphate | High in |
| | | reductase subunit M2; Ribonucleotide | Severe |
| | | reductase small chain; | |
| | | Ribonucleotide reductase small | |
| | | subunit | |
| GRPEL1 | Q9HAV7 | GrpE protein homolog 1, | High in |
| | | GrpE protein homolog 1, | Severe |
| | | mitochondrial; HMGE; Mt-GrpE#1 | |
| TRIM21 | P19474 | E3 ubiquitin-protein ligase TRIM21; | High in |
| | | 52 kDa Ro protein; | Severe |
| | | 52 kDa ribonucleoprotein autoantigen | |
| | | Ro/SS-A; | |
| | | RING finger protein 81; | |
| | | RING-type E3 ubiquitin transferase; | |
| | | TRIM21 Curated; | |
| | | Ro(SS-A) | |
| DDX58 | O95786 | Antiviral innate immune response | High in |
| | | receptor RIG-I; | Severe |
| | | ATP-dependent RNA helicase | |
| | | DDX58; | |
| | | DEAD box protein 58; | |
| | | RIG-I-like receptor 1 (RLR-1); | |
| | | RNA sensor RIG-I; | |
| | | Retinoic acid-inducible gene 1 protein | |
| | | (RIG-1) | |
| KRT18 | P05783 | Keratin, type I cytoskeletal 18; | High in |
| | | Cell proliferation-inducing gene 46 | Severe |
| | | protein; | |
| | | Cytokeratin-18 (CK-18); | |
| | | Keratin-18 (K18) | |
| AGER/ | Q15109 | AGER (Advanced glycosylation end | High in |
| RAGE | | product-specific receptor); Receptor | Severe |
| | | for advanced glycosylation end | |
| | | products | |
| SIT1 | Q9Y3P8 | Signaling threshold-regulating | High in |
| | | transmembrane adapter 1; | Severe |
| | | SHP2-interacting transmembrane | |
| | | adapter protein; | |
| | | Suppression-inducing transmembrane | |
| | | adapter 1; | |
| | | gp30/40 | |
| CRTAC1 | Q9NQ79 | Cartilage acidic protein 1; | Low in |
| | | 68 kDa chondrocyte-expressed protein | Severe |
| | | (CEP-68); | |
| | | ASPIC | |
| CDON | Q4KMG0 | Cell adhesion molecule- | High in |
| | | related/down-regulated by oncogenes | Severe |
| CCL17 | Q92583 | C-C motif chemokine 17; | High in |
| | | CC chemokine TARC; | Severe |
| | | Small-inducible cytokine A17; | |
| | | Thymus and activation-regulated | |
| | | chemokine | |
| TNFRSF10C | O14798 | Tumor necrosis factor receptor | High in |
| | | superfamily member 10C; | Severe |
| | | Antagonist decoy receptor for TRAIL | |
| | | Apo-2L; | |
| | | Decoy TRAIL receptor without death | |
| | | domain; | |
| | | Decoy receptor 1 (DcR1); | |
| | | Lymphocyte inhibitor of TRAIL; | |
| | | TNF-related apoptosis-inducing | |
| | | ligand receptor 3 (TRAIL receptor 3; | |
| | | TRAIL-R3) | |
| CD1C | P29017 | CD1C (T-cell surface glycoprotein | Low in |
| | | CD1c) | Severe |
| DSC2 | Q02487 | Desmocollin-2 | High in |
| | | | Severe |
| FCGR3B | O75015 | Low affinity immunoglobulin gamma | Low in |
| | | Fc region receptor III-B; | Severe |
| | | Fc-gamma RIII-beta; | |
| | | FcR-10; | |
| | | IgG Fc receptor III-1 | |
| QPCT | Q16769 | Glutaminyl-peptide cyclotransferase; | High in |
| | | Glutaminyl cyclase (QC; sQC); | Severe |
| | | Glutaminyl-tRNA cyclotransferase; | |
| | | Glutamyl cyclase (EC) | |
| TNXB | P22105 | Tenascin-X; TN-X; Hexabrachion- | High in |
| | | like protein | Severe |
| ADAM15 | Q13444 | Disintegrin and metalloproteinase | High in |
| | | domain-containing protein 15; | Severe |
| | | Metalloprotease RGD disintegrin | |
| | | protein; | |
| | | Metalloproteinase-like, disintegrin- | |
| | | like, and cysteine-rich protein 15 | |
| | | (MDC-15); Metargidin | |
| AREG | P15514 | AREG (Amphiregulin); CRDGF | High in |
| | | | Severe |
| ASAH2 | Q9NR71 | Neutral ceramidase | Low in |
| | | | Severe |
| CBLN4 | Q9NTU7 | Cerebellin-4; Cerebellin-like | Low in |
| | | glycoprotein 1 | Severe |
| CCL24 | O00175 | Eotaxin-2; MPIF-2; CK-beta-6; | Low in |
| | | CCL24 | Severe |
| CCL8 | P80075 | C-C motif chemokine 8; HC14; | High in |
| | | Monocyte chemoattractant protein 2; | Severe |
| | | Monocyte chemotactic protein 2 | |
| | | (MCP-2); | |
| | | Small-inducible cytokine A8 | |
| CDH5 | P33151 | Cadherin-5; 7B4 antigen | High in |
| | | | Severe |
| CTSB | P07858 | CTSB (Cathepsin B); APPS | High in |
| | | | Severe |
| CTSL | P07711 | CTSL (Procathepsin L) (Cathepsin | High in |
| | | L1); MEP | Severe |
| MATN3 | O15232 | MATN3 (Matrilin-3) | High in |
| | | | Severe |
| MPHOSPH8 | Q99549 | MPHOSPH8 (M-phase | High in |
| | | phosphoprotein 8); Twa3 | Severe |
| NADK | 095544 | NAD kinase | High in |
| | | | Severe |
| P4HB | P07237 | P4HB; PDI (Protein disulfide- | High in |
| | | isomerase); p55 | Severe |
| PLAUR/ | Q03405 | soluble u-PAR; Urokinase | High in |
| MO3/UPAR | | plasminogen activator surface | Severe |
| | | receptor; U-PAR; uPAR; suPAR; | |
| | | Monocyte activation antigen Mo3; | |
| | | CD87; | |
| PQBP1 | O60828 | PQBP1 (Polyglutamine-binding | High in |
| | | protein 1); Npw38 | Severe |
| REGIA | P05451 | PSP (pancreatic stone protein) | High in |
| | | | Severe |
| REG1B | P48304 | PSP-2; Lithostathine-1-beta | High in |
| | | | Severe |
| SIGLEC6 | O43699 | Sialic acid-binding Ig-like lectin 6; | Low in |
| | | Siglec-6; CD33 antigen-like 1 | Severe |
| TNFRSF6B | O95407 | DcR3(TNFRSF6B) | High in |
| | | | Severe |
| TRIAP1 | O43715 | TRIAP1 (TP53-regulated inhibitor of | High in |
| | | apoptosis 1); WF-1; p53CSV | Severe |
| UMOD | P07911 | UMOD (Uromodulin); THP | Low in |
| | | | Severe |
| TRAIL/ | P50591 | Tumor necrosis factor ligand | Low in |
| TNFSF10 | | superfamily member 10; Apo-2 ligand | Severe |
| | | (Apo-2L); Protein TRAIL | |
| CRP | P02741 | C-reactive protein | High in |
| | | | Severe |
| CXCL10 | P02778 | C-X-C motif chemokine 10; IP-10 | High in |
| | | | Severe |

Table 7 lists proteins that were found to be differentially expressed in infectious vs. non-infectious etiologies with a high AUC.

TABLE 7

| | UniProt | | |
|---|---|---|---|
| Gene Symbol | ID | Biomarker | Direction |
| PM20D1 | Q6GTS8 | N-fatty-acyl-amino acid synthase; | High in Infectious |
| | | hydrolase PM20D1; | |
| | | Peptidase M20 domain-containing protein | |
| | | 1 | |
| IFNG | P01579 | Interferon gamma; IFN-gamma; Immune | High in Infectious |
| | | interferon | |
| IL-10 | P22301 | Interleukin-10; IL-10; Cytokine synthesis | High in Infectious |
| | | inhibitory factor (CSIF) | |
| DDX58 | O95786 | Antiviral innate immune response | High in Infectious |
| | | receptor RIG-I; ATP-dependent RNA | |
| | | helicase DDX58; DEAD box protein 58 | |
| | | RIG-I-like receptor 1 (RLR-1) | |
| | | RNA sensor RIG-I; Retinoic acid- | |
| | | inducible gene 1 protein (RIG-1) | |
| CXCL11 | O14625 | C-X-C motif chemokine 11; Beta-R1 | High in Infectious |
| | | H174; Interferon gamma-inducible protein | |
| | | 9 (IP-9); | |
| | | Interferon-inducible T-cell alpha | |
| | | chemoattractant (I-TAC); | |
| | | Small-inducible cytokine B11 | |
| SIGLEC5 | O15389 | Sialic acid-binding Ig-like lectin 5; Siglec- | High in Infectious |
| | | 5; CD33 antigen-like 2; | |
| | | Obesity-binding protein 2 (OB-BP2; OB- | |
| | | binding protein 2) | |
| NADK | O95544 | NAD kinase; Poly(P); ATP NAD kinase | High in Infectious |
| CCL8 | P80075 | C-C motif chemokine 8; HC14 | High in Infectious |
| | | Monocyte chemoattractant protein 2; | |
| | | Monocyte chemotactic protein 2 (MCP- | |
| | | 2); | |
| | | Small-inducible cytokine A8 | |
| PPP1R9B | Q96SB3 | Neurabin-2; Neurabin-II; | High in Infectious |
| | | Protein phosphatase 1 regulatory subunit | |
| | | 9B/Spinophilin | |
| SIGLEC1 | Q9BZZ2 | Sialoadhesin; Sialic acid-binding Ig-like | High in Infectious |
| | | lectin 1 (Siglec-1) | |
| IL6/IFNB2 | P05231 | Interleukin-6; IL-6; B-cell stimulatory | High in Infectious |
| | | factor 2 (BSF-2); CTL differentiation | |
| | | factor (CDF); Hybridoma growth factor | |
| | | Interferon beta-2 (IFN-beta-2) | |
| PLA2G2A | P14555 | sPLA2-IIA (Group IIA phospholipase | High in Infectious |
| | | A2); NPS-PLA2 | |
| CSF3 | P09919 | CSF3; G-CSF (Granulocyte colony- | High in Infectious |
| | | stimulating factor); Pluripoietin; | |
| | | Filgrastim; Lenograstim | |
| PRTN3 | P24158 | proteinase 3/Myeloblastin | High in Infectious |
| MMP8 | P22894 | MMP 8; PMNL-CL | High in Infectious |
| LBP | P18428 | LBP (Lipopolysaccharide binding | High in Infectious |
| | | protein) | |
| VWA1 | Q6PCB0 | von Willebrand factor A domain- | High in Infectious |
| | | containing protein 1 | |
| OSM | P13725 | Oncostatin-M | High in Infectious |
| GPR37 | O15354 | Prosaposin receptor GPR37; Endothelin B | High in Infectious |
| | | receptor-like protein 1 (ETBR-LP-1)/ | |
| | | G-protein coupled receptor 37; | |
| | | Parkin-associated endothelin receptor-like | |
| | | receptor (PAELR) | |
| IL1RN | P18510 | IL-1ra/Anakinra/Interleukin-1 receptor | High in Infectious |
| | | antagonist protein | |
| CXCL10 | P02778 | C-X-C motif chemokine 10/IP-10 | High in Infectious |
| CRP | P02741 | C-reactive protein | High in Infectious |

### Example 2

A second study was carried out to uncover particular markers useful for determining severity of an infectious disease.

### Study Details

### Inclusion Criteria:

### Suspected Acute Infection Patients

Over 18 years of age
Clinical suspicion of acute infection as defined by the attending physician, based on clinical presentation.

### Healthy Individuals

Over 18 years of age
No clinical suspicion of acute infection

### Exclusion Criteria:

### Suspected Acute Infection Patients:

Patients fulfilling the following criteria were not eligible for inclusion in this study:
HIV, HBV, active HCV or active Tuberculosis infection (self-declared or known from medical records)
Pregnancy—self reported or medically confirmed

### Healthy Individuals

Patients fulfilling the following criteria are not eligible for inclusion in this study:
Episode of infection in the last 2 weeks
Major trauma and\or burns and\or surgery in the last 2 weeks
HIV, HBV, active HCV or active Tuberculosis infection (self-declared or known from medical records)
Elective surgery patients
Pregnancy—self reported or medically confirmed

Protein screening was performed using 2 multiplex immunoassays: Human Magnetic Luminex® Assays and RayBiotech Custom Quantibody® Human Arrays, and 4 single ELISAs. In total, 54 proteins were measured providing absolute protein concentrations. Study cohort included 247 patients, out of which 87 severe and 160 non-severe patients, see Table 8. In addition, MR-proADM was measured using B⋅R⋅A⋅H⋅M⋅S MR-proADM KRYPTOR assay on a subset of the cohort (44 severe. 75 non-severe patients).

TABLE 8

| Parameter | | Statistics | Cohort | Non-severe | Severe |
|---|---|---|---|---|---|
| Demographics | Age (years) | median | 72.3 (61.5, 83.1) | 72.0 (59.8, 82.0) | 76.0 (62.5, 86.0) |
| | | (IQR) | | | |
| | Sex: male | n (%) | 161 (65.2%) | 102 (63.8%) | 59 (67.8%) |
| Source of | LRTI | n (%) | 121 (49.0%) | 69 (43.1%) | 52 (59.8%) |
| infection | URTI | n (%) | 5 (2.0%) | 4 (2.5%) | 1 (1.1%) |
| | Urinary | n (%) | 60 (24.3%) | 43 (26.9%) | 17 (19.5%) |
| | GI | n (%) | 23 (9.3%) | 15 (9.4%) | 8 (9.2%) |
| | Bacteremia | n (%) | 12 (4.9%) | 9 (5.6%) | 3 (3.4%) |
| | Soft tissue | n (%) | 21 (8.5%) | 13 (8.1%) | 8 (9.2%) |
| | Unknown | n (%) | 8 (3.2%) | 3 (1.9%) | 5 (5.7%) |
| | source | | | | |
| Adjudication | Bacterial | n (%) | 136 (55.1%) | 90 (56.2%) | 46 (52.9%) |
| | Non-infectious | n (%) | 20 (8.1%) | 17 (10.6%) | 3 (3.4%) |
| | Viral | n (%) | 41 (16.6%) | 26 (16.2%) | 15 (17.2%) |

Disease etiology was established by applying a rigorous expert panel adjudication process.

All patients had a blood sample taken during their hospitalization or emergency department visit course. Comparisons between the patient groups were carried out for identifying differential markers in blood serum.

National Early Warning Score, (NEWS) was calculated for a sub set of the cohort (62 severe, 121 non-severe patients).

Severe patients were defined as those who died within 14 days from blood draw, or met any of the following outcomes within 3 days from blood draw:
Vasopressor therapy
Intubation with mechanical ventilation
Non-invasive ventilation
Admission to the intensive care unit (ICU)

Patients who did not meet any of the above outcomes were defined as non-severe.

### Measures of Biomarker Performance:

Performance measures for differentia ting between severe and non-severe groups included sensitivity (for detecting severe patients) arid specificity, at 2 cutoffs:
Rule-out cutoff: determined based on required sensitivity of 90%
Rule-in cutoff: determined based on required specificity of 80%
   Performance of combinations of multiple markers is based on the probabilities from a logistic regression model.

### Results

Table 9 summarizes the results of relevant proteins in terms of their ability to either rule in or rule out a severe infection using particular cut-offs.

TABLE 9

| | rule out | | | rule in | | |
|---|---|---|---|---|---|---|
| | cutoff | sen- | spec- | cutoff | sen- | spec- |
| Name | (pg/ml) | sitivity | ificity | (pg/ml) | sitivity | ificity |
| TSG-14 | 930 | 90% | 26% | 6,000 | 59% | 80% |
| AGER | 960 | 90% | 8% | 3,200 | 57% | 80% |
| ANG-2 | 1800 | 90% | 23% | 5000 | 55% | 80% |
| ST2 | 280,00 | 90% | 36% | 140,000 | 54% | 80% |
| IL-10 | 0.17 | 89% | 0% | 68 | 53% | 80% |
| IL-6 | 9.8 | 90% | 24% | 56 | 45% | 80% |

Table 10 summarizes the results of pairs of proteins in terms of their ability to either rule in or rule out a severe infection based on the probabilities from a logistic regression model. The pair AGER+ANG-2 show improved in performances as compared to the single markers. The pairs AGER+ST2 and ST2+ANG-2 show improved in performances for “rule in”

TABLE 10

| marker 1 name | marker 2 name | purpose | sensitivity | specificity |
|---|---|---|---|---|
| AGER | ANG-2 | rule out | 90% | 31% |
| ST2 | AGER | rule out | 90% | 27% |
| ST2 | ANG-2 | rule out | 90% | 29% |
| AGER | ANG-2 | rule in | 67% | 80% |
| ST2 | AGER | rule in | 66% | 80% |
| ST2 | ANG-2 | rule in | 57% | 80% |

Table 11 summarizes the results of using AGER and ANG-2 as single markers or as a pair of markers for determining severity in subgroups of subjects or using different definitions for severity.

TABLE 11

| | | AGER | | ANG-2 | | pair | |
|---|---|---|---|---|---|---|---|
| Task | Purpose | sensitivity | specificity | sensitivity | specificity | sensitivity | specificity |
| Entire cohort | rule out | 90% | 8% | 90% | 23% | 90% | 31% |
| Viral etiology | rule out | 87% | 42% | 87% | 12% | 87% | 46% |
| Baseline severity NEWS >=7 | rule out | 89% | 0% | 89% | 24% | 89% | 28% |
| Age <65 years | rule out | 88% | 7% | 88% | 30% | 88% | 41% |
| Age >=65 and <80 years | rule out | 89% | 6% | 89% | 31% | 89% | 35% |
| Age >=80 | rule out | 89% | 29% | 89% | 23% | 89% | 37% |
| Met a severe outcome 2-3 | rule out | 87% | 4% | 87% | 9% | 87% | 29% |
| days after blood draw | | | | | | | |
| Entire cohort | rule in | 57% | 80% | 55% | 80% | 67% | 80% |
| Viral etiology | rule in | 67% | 77% | 53% | 77% | 73% | 77% |
| Baseline severity NEWS 0-4 | rule in | 43% | 80% | 43% | 80% | 57% | 80% |
| Baseline severity NEWS 5-6 | rule in | 50% | 77% | 55% | 77% | 70% | 77% |
| Baseline severity NEWS >=7 | rule in | 46% | 80% | 46% | 80% | 64% | 80% |
| Age <65 years | rule in | 58% | 80% | 58% | 80% | 67% | 80% |
| Age >=65 and <80 years | rule in | 57% | 80% | 46% | 80% | 64% | 80% |
| Met a severe outcome on | rule in | 57% | 80% | 76% | 80% | 81% | 80% |
| the day of blood draw | | | | | | | |
| (“same day”) | | | | | | | |
| Met a severe outcome 2-3 | rule in | 61% | 80% | 43% | 80% | 65% | 80% |
| days after blood draw | | | | | | | |
| Time from symptom onset: | rule in | 56% | 78% | 56% | 78% | 69% | 78% |
| 2-3 days | | | | | | | |
| Time from symptom onset: | rule in | 33% | 80% | 50% | 80% | 75% | 80% |
| 4-7 days | | | | | | | |
| “Severe” defined as | rule in | 47% | 80% | 53% | 80% | 58% | 80% |
| intubation with mechanical | | | | | | | |
| ventilation (IMV) within 3 | | | | | | | |
| days after blood draw as | | | | | | | |
| a proxy for respiratory | | | | | | | |
| failure | | | | | | | |

Table 12 summarizes the results of using ST2 and ANG-2 as single markers or as a pair of markers for determining severity in subgroups of subjects or using different definitions of severity.

| | | ST2 | | ANG-2 | | pair | |
|---|---|---|---|---|---|---|---|
| Task | Purpose | sensitivity | specificity | sensitivity | specificity | sensitivity | specificity |
| Bacterial etiology | rule out | 89% | 29% | 89% | 32% | 89% | 46% |
| Baseline severity NEWS >=7 | rule out | 89% | 36% | 89% | 24% | 89% | 44% |
| Age >=80 | rule out | 89% | 38% | 89% | 23% | 89% | 40% |
| Died 4-14 days after | rule out | 83% | 28% | 83% | 55% | 83% | 61% |
| blood draw (time to | | | | | | | |
| outcome is 4-14 days) | | | | | | | |
| Time from symptom | rule out | 87% | 32% | 87% | 43% | 87% | 55% |
| onset: 0-1 days | | | | | | | |
| “Severe” defined as | rule out | 90% | 47% | 90% | 36% | 90% | 67% |
| Vasopressor therapy | | | | | | | |
| within 3 days after blood | | | | | | | |
| draw as a proxy for | | | | | | | |
| septic shock | | | | | | | |
| “Severe” defined as | rule out | 88% | 33% | 88% | 43% | 88% | 54% |
| death within 14 days | | | | | | | |
| after blood draw | | | | | | | |
| Entire cohort | rule in | 54% | 80% | 55% | 80% | 57% | 80% |
| Bacterial etiology | rule in | 65% | 80% | 65% | 80% | 67% | 80% |
| Baseline severity NEWS 0-4 | rule in | 57% | 80% | 43% | 80% | 64% | 80% |
| Baseline severity NEWS >=7 | rule in | 54% | 80% | 46% | 80% | 57% | 80% |
| Met a severe outcome on | rule in | 71% | 80% | 76% | 80% | 81% | 80% |
| the day of blood draw | | | | | | | |
| (“same day”) | | | | | | | |
| “Severe” defined as | rule in | 70% | 80% | 73% | 80% | 80% | 80% |
| vasopressor therapy | | | | | | | |
| within 3 days after blood | | | | | | | |
| draw as a proxy for | | | | | | | |
| septic shock | | | | | | | |
| “Severe” defined as | rule in | 53% | 80% | 53% | 80% | 58% | 80% |
| intubation with | | | | | | | |
| mechanical ventilation | | | | | | | |
| (IMV) within 3 days | | | | | | | |
| after blood draw as a | | | | | | | |
| proxy for respiratory | | | | | | | |
| failure | | | | | | | |

Table 13 summarizes the results of using AGER and ST2 as single markers or as a pair of markers for determining severity in subgroups of subjects or using different definitions of severity.

| | | ST2 | | AGER | | pair | |
|---|---|---|---|---|---|---|---|
| Task | Purpose | sensitivity | specificity | sensitivity | specificity | sensitivity | specificity |
| Baseline severity NEWS 5-6 | rule out | 90% | 32% | 90% | 45% | 90% | 52% |
| Age >=80 years | rule out | 89% | 38% | 89% | 29% | 89% | 60% |
| Died 4-14 days after | rule out | 83% | 28% | 83% | 46% | 83% | 66% |
| blood draw (time to | | | | | | | |
| outcome is 4-14 days) | | | | | | | |
| Time from symptom | rule out | 87% | 32% | 87% | 11% | 87% | 34% |
| onset: 0-1 days | | | | | | | |
| Time from symptom | rule out | 83% | 40% | 83% | 40% | 83% | 69% |
| onset: 4-7 days | | | | | | | |
| “Severe” defined as | rule out | 90% | 47% | 90% | 31% | 90% | 51% |
| vasopressor therapy | | | | | | | |
| within 3 days after blood | | | | | | | |
| draw as a proxy for | | | | | | | |
| septic shock | | | | | | | |
| “Severe” defined as | rule out | 89% | 29% | 89% | 39% | 89% | 46% |
| intubation with | | | | | | | |
| mechanical ventilation | | | | | | | |
| (IMV) within 3 days | | | | | | | |
| after blood draw as a | | | | | | | |
| proxy for respiratory | | | | | | | |
| failure | | | | | | | |
| “Severe” defined as death | rule out | 88% | 33% | 88% | 33% | 88% | 57% |
| within 14 days after | | | | | | | |
| blood draw | | | | | | | |
| Entire cohort | rule in | 54% | 80% | 57% | 80% | 66% | 80% |
| Bacterial etiology | rule in | 65% | 80% | 48% | 80% | 70% | 80% |
| Baseline severity NEWS 0-4 | rule in | 57% | 80% | 43% | 80% | 71% | 80% |
| Baseline severity NEWS >=7 | rule in | 54% | 80% | 46% | 80% | 75% | 80% |
| Age <65 years | rule in | 58% | 80% | 58% | 80% | 75% | 80% |
| Age >=65 and <80 years | rule in | 39% | 80% | 57% | 80% | 64% | 80% |
| Met a severe outcome on | rule in | 71% | 80% | 57% | 80% | 81% | 80% |
| the day of blood draw | | | | | | | |
| (“same day”) | | | | | | | |
| Met a severe outcome | rule in | 48% | 80% | 52% | 80% | 56% | 80% |
| one day after blood draw | | | | | | | |
| Met a severe outcome 2-3 | rule in | 43% | 80% | 61% | 80% | 70% | 80% |
| days after blood draw | | | | | | | |
| Time from symptom | rule in | 70% | 79% | 52% | 79% | 83% | 79% |
| onset: 0-1 days | | | | | | | |
| Time from symptom | rule in | 58% | 80% | 33% | 80% | 75% | 80% |
| onset: 4-7 days | | | | | | | |
| Time from symptom | rule in | 60% | 79% | 60% | 79% | 70% | 79% |
| onset: >7 days | | | | | | | |
| “Severe” defined as | rule in | 53% | 80% | 47% | 80% | 58% | 80% |
| intubation with | | | | | | | |
| mechanical ventilation | | | | | | | |
| (IMV) within 3 days | | | | | | | |
| after blood draw as a | | | | | | | |
| proxy for respiratory | | | | | | | |
| failure | | | | | | | |

TABLE 14

| | NEWS | | | AGER + ANG-2 | |
|---|---|---|---|---|---|
| Purpose | cutoff | sensitivity | specificity | sensitivity | specificity |
| rule in | >6 | 45% | 79% | 67% | 80% |
| | NEWS | | | AGER + ST2 | |
| Purpose | cutoff | sensitivity | specificity | sensitivity | specificity |
| rule in | >6 | 45% | 79% | 66% | 80% |
| | NEWS | | | ANG-2 + ST2 | |
| Purpose | cutoff | sensitivity | specificity | sensitivity | specificity |
| rule in | >6 | 45% | 79% | 57% | 80% |

The ability of the marker MR-proADM to predict severity of infection was also analyzed. As shown in Table 15, together with additional determinants, this marker showed that it was useful for determining severity of infection

TABLE 15

| | MR-proADM | | | IP-10 | | | | |
|---|---|---|---|---|---|---|---|---|
| | Cutoff | | | Cutoff | | | MR-proADM + IP-10 | |
| Purpose | (nmol/L) | sensitivity | specificity | (pg/ml) | sensitivity | specificity | sensitivity | specificity |
| rule out | 0.6 | 89% | 16% | 100 | 68% | 0% | 89% | 39% |
| rule in | 1.9 | 52% | 80% | 565 | 30% | 79% | 59% | 80% |
| | MR-proADM | | | CRP | | | | |
| | Cutoff | | | Cutoff | | | MR-proADM + CRP | |
| Purpose | (nmol/L) | sensitivity | specificity | (mg/L) | sensitivity | specificity | sensitivity | specificity |
| rule out | 0.6 | 89% | 16% | 23.4 | 89% | 24% | 89% | 29% |

| | MR-proADM | | | AGER | | | | |
|---|---|---|---|---|---|---|---|---|
| | Cutoff | | | Cutoff | | | MR-proADM + AGER | |
| Purpose | (nmol/L) | sensitivity | specificity | (pg/ml) | sensitivity | specificity | sensitivity | specificity |
| rule out | 0.6 | 89% | 16% | 957.2 | 90% | 8% | 89% | 33% |
| rule in | 1.9 | 52% | 80% | 3,163.30 | 57% | 80% | 61% | 80% |

As summarized in Table 16, IP10 improved the ability of particular markers to determine the severity of infectious diseases.

TABLE 16

| | Marker 1 | | | Marker 2 | | | pair | |
|---|---|---|---|---|---|---|---|---|
| Purpose | analyte | sensitivity | specificity | analyte | sensitivity | specificity | sensitivity | specificity |
| rule out | IP-10 | 68% | 0% | AGER | 90% | 8% | 90% | 15% |
| rule in | IP-10 | 30% | 79% | AGER | 57% | 80% | 62% | 80% |
| rule out | IP-10 | 68% | 0% | ANG-2 | 90% | 23% | 90% | 33% |
| rule in | IP-10 | 30% | 79% | ANG-2 | 55% | 80% | 60% | 80% |
| rule out | IP-10 | 68% | 0% | IL-10 | 89% | 0% | 90% | 21% |
| rule in | IP-10 | 30% | 79% | ST2 | 54% | 80% | 61% | 80% |
| rule out | IP-10 | 68% | 0% | TSG-14 | 90% | 26% | 90% | 31% |

As summarized in Table 17, IP-10 improved the ability of particular pairs to determine the severity of infectious diseases.

TABLE 17

| | IP-10 | | AGER | | ANG-2 | | IP-10 + AGER + ANG-2 | |
|---|---|---|---|---|---|---|---|---|
| Purpose | sensitivity | specificity | sensitivity | specificity | sensitivity | specificity | sensitivity | specificity |
| rule out | 68% | 0% | 90% | 8% | 90% | 23% | 90% | 35% |
| rule in | 30% | 79% | 57% | 80% | 55% | 80% | 69% | 80% |

| | | | |
|---|---|---|---|
| IP-10 | ST2 | ANG-2 | IP-10 + ST2 + ANG-2 |

| Purpose | sensitivity | specificity | sensitivity | specificity | sensitivity | specificity | sensitivity | specificity |
|---|---|---|---|---|---|---|---|---|
| rule in | 30% | 79% | 54% | 80% | 55% | 80% | 59% | 80% |

### Example 3

### Materials and Methods

The study cohort was comprised of 261 COVID-19 patients that were recruited prospectively in 37 study sites (29 in Greece and eight in Italy) as part of a double-blind randomized study. Of the 261 patients, 167 (64.0%) were male and 188 (73.2%) suffered from severe pneumonia according to WHO classification. The average age was 55.5 years and average BMI was 25.7. All patient in this cohort were treated according to the standard of care guidelines at time of treatment. Of note, 206 patients (78.9%) were treated with Dexamethasone during the trial.

Sample measurement: suPAR levels were measured using an ELISA assay. IP-10 levels were measured using MeMed Key™ platform.

A severe outcome was defined as severe respiratory failure (SRF) or mortality within 14 days from blood draw. SRF was defined as a respiratory ratio (partial oxygen pressure (PaO2)/fraction of inspired oxygen (FiO2)) below 150 mmHg, necessitating non-invasive ventilation (NIV) or mechanical ventilation (MV). 15% of cohort patients met a severe outcome.

### Results

The combination of IP-10 and suPAR was shown to accurately distinguish between severe and non-severe outcome, as summarized in Table 18.

TABLE 18
IP-10 and suPAR single/doublet's accuracy for distinguishing
between severe and non-severe outcome.

| Analyte | AUC |
|---|---|
| suPAR | 0.80 |
| IP-10 | 0.81 |
| IP-10 + suPAR | 0.85 |

The combination of IL-6 and suPAR was shown to accurately distinguish between severe and non-severe outcome, as summarized in Table 19.

TABLE 19
IL-6 and suPAR single/doublet's accuracy for distinguishing
between severe and non-severe outcome.

| Analyte | AUC |
|---|---|
| suPAR | 0.80 |
| IL-6 | 0.78 |
| IL-6 + suPAR | 0.83 |

In addition, any priority document(s) of this application is/are hereby incorporated herein by reference in its/their entirety.

1. A method of treating a subject having an infectious disease comprising:
(a) measuring an expression level of at least one protein selected from the group consisting of Tumor necrosis factor-inducible gene 14 protein (TSG-14), Advanced glycosylation end product-specific receptor (AGER), Angiogpoietin-2 (ANG-2) and -Interleukin 1 receptor-like 1 (ST2) in a sample of the subject so as to diagnose the infectious disease based on said expression level; and
(b) treating the subject according to the diagnosis of the infection.

2. The method of claim 1, wherein
(i) when said expression level of TSG-14 is at least 2 fold higher than a level in a control sample, a severe infectious disease is ruled in;
(ii) when said expression level of AGER is at least 2 fold higher than a level in a control sample, a severe infectious disease is ruled in;
(iii) when said expression level of ANG-2 is at least 2 fold higher than a level in a control sample, a severe infectious disease is ruled in; and/or
(iv) when said expression level of ST2 is at least 3 fold higher than a level in a control sample, a severe infectious disease is ruled in.

3-5. (canceled)

6. The method of claim 1, wherein said at least one protein comprises at least two proteins selected from the group consisting of ANG-2 and AGER; AGER and ST2; or ANG-2 and ST2.

7. The method of claim 1, further comprising measuring an expression of at least one additional protein selected from the group consisting of IL-6, IL-10, suPAR, DR5 and MR-proADM and diagnosing the infectious disease based on said expression level of said at least one additional protein in combination with said expression level of said at least one protein.

8-9. (canceled)

10. The method of claim 7, wherein:
(i) when said expression level of IL-10 is at least 3 fold higher than a level in a control sample, a severe infectious disease is ruled in;
(ii) when said expression level of IL-6 is is at least 2 fold higher than a level in a control sample, a severe infectious disease is ruled in.

11. The method of claim 1, further comprising measuring an expression level of IP-10 and diagnosing the infection based on said expression level of IP-10 in combination with said expression level of said at least one protein.

12. The method of claim 6, further comprising measuring an expression level of IP-10 and diagnosing the infection based on said expression level of IP-10 in combination with said expression level of said at least two proteins.

13-17. (canceled)

18. The method of claim 1, further comprising measuring an expression level of TRAIL and/or CRP.

19. The method of claim 1, further comprising measuring all the components of a clinical index selected from the group consisting of NEWS, NEWS 2, MEWS, ESI, APACHE I, APACHE II, APACHE III, CURB-65, SMART-COP, SAPS II, SAPS III, PIM2, CMM, SOFA, qSOFA, MPM, RIFLE, CP, MODS, LODS, Rochester criteria, Philadelphia Criteria, Milwaukee criteria and Ranson score.

20. The method of claim 1, further comprising measuring the level of at least one additional protein set forth in Tables 5, 6 or 7.

21. The method of claim 1, wherein the infection is a viral infection.

22. The method of claim 1, wherein the infection is a bacterial infection.

23. The method of claim 1, wherein the subject shows symptoms of an infectious disease.

24. (canceled)

25. The method of claim 1, wherein the subject does not have a chronic non-infectious disease.

26-28. (canceled)

29. The method of claim 1, wherein the level of no more than 10 proteins is used to diagnose the infection.

30. (canceled)

31. The method of claim 1, wherein said diagnosing an infection comprises determining a severity of the infection.

32. (canceled)

33. A kit for diagnosing an infection comprising detection reagents which specifically at least two determinants selected from the group consisting of TSG-14, AGER, ANG-2 and ST2.

34-35. (canceled)

36. The kit of claim 33, further comprising a detection reagent which specifically detects IP-10.

37-42. (canceled)

43. The kit of claim 33, wherein said kit comprises detection reagents that specifically detect no more than 10 protein markers.

44-45. (canceled)

46. The method of claim 1, wherein when a severe infection is ruled in, at least one of the following treatments is used: hospitalization; placement in intensive care; mechanical ventilation; non-invasive ventilation, ECMO, renal replacement therapy, cardiac catheterization, Antibiotic treatment, vasopressor therapy and/or treatment of last resort.

47-48. (canceled)
